(19) United States
(12) Patent Application Publication
(10) Pub. No.: US 2023/0010287 A1
(43) Pub. Date: Jan. 12, 2023

(54) PAIRED-END SEQUENCING METHODS AND COMPOSITIONS

(71) Applicant: Pacific Biosciences of California, Inc., Menlo Park, CA (US)

(72) Inventors: Jeremiah Hanes, Menlo Park, CA (US); Robert H. Reamey, Menlo Park, CA (US); Lin Wang, Menlo Park, CA (US); Fabian Block, Menlo Park, CA (US); Kurt Patterson, Menlo Park, CA (US); Brittany Ann Rohrman, Menlo Park, CA (US); Andrew Sparks, Menlo Park, CA (US); Evan Hurowitz, Menlo Park, CA (US); Joan Wilson, Menlo Park, CA (US); Min-Jui Richard Shen, Menlo Park, CA (US)

(21) Appl. No.: 17/859,770

(22) Filed: Jul. 7, 2022

Related U.S. Application Data
(60) Provisional application No. 63/219,738, filed on Jul. 8, 2021.
(60) Provisional application No. 63/246,188, filed on Sep. 20, 2021.

(51) Int. Cl. C12Q 1/6869

(52) U.S. Cl. CPC C12Q 1/6869; C12Q 1/6855

(57) ABSTRACT

The present invention provides methods and compositions for carrying out nucleic acid sequencing, particularly paired-end sequencing. The methods use concatemeric sequencing templates that can be produced by rolling circle amplification of asymmetric circular nucleic acids having a central double-stranded region comprising a target nucleic acid sequence that is connected at each end to form a circular construct.

### CROSS-REFERENCE TO RELATED APPLICATIONS

This application is a non-provisional utility patent application claiming priority to and benefit of the following prior provisional patent applications: U.S. Ser. No. 63/246,188, filed Sep. 20, 2021, entitled “PAIRED-END SEQUENCING METHODS AND COMPOSITIONS” by Jeremiah Hanes et al., and U.S. Ser. No. 63/219,738, filed Jul. 8, 2021, entitled “PAIRED-END SEQUENCING METHODS AND COMPOSITIONS” by Jeremiah Hanes et al. Each of these applications is incorporated herein by reference in its entirety for all purposes.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Development of nucleic acid sequencing technologies has yielded countless advances in numerous areas. The ability to rapidly and reliably determine the sequence of DNA and RNA molecules has enabled numerous advances in molecular biology, evolutionary biology, medical diagnostics, and molecular medicine, among many other fields.

The amount of sequence data that can be reliably obtained when using certain sequencing by synthesis or sequencing by binding techniques, however, may be limited to a relatively small number of bases. While short sequence reads can be extremely useful in applications such as, for example, SNP analysis and genotyping, in many circumstances it can be advantageous to be able to reliably obtain further sequence data for the same template molecule. To this end, paired-end or pairwise sequencing techniques have been employed, e.g., particularly in the context of whole genome shotgun sequencing. Paired-end sequencing can allow the determination of two “reads” of sequence from two places on a single polynucleotide duplex. The knowledge that the paired-end sequences are known to occur on a single duplex, and are therefore linked or paired in the genome, can greatly aid assembly of whole genome sequences into a consensus sequence. The additional information obtained from paired-end sequencing can also benefit other applications, e.g., applications involving sequencing cell-free DNA such as detection of circulating tumor DNA and prenatal cell-free DNA screening.

Provided here are novel and useful compositions and methods for carrying out paired-end sequencing. These compositions and methods provide advantages to a number of sequencing methods, e.g., stepwise sequencing methods.

### SUMMARY OF THE INVENTION

One general class of embodiments provides methods for paired-end sequencing. In the methods, a nucleic acid concatemer is provided that comprises multiple sequential copies of: a first adapter region, a forward strand of a target nucleic acid sequence, a second adapter region different from the first adapter region, and a reverse strand of the target nucleic acid sequence that is complementary to the forward strand. A sequencing process is performed to produce paired-end reads of the target nucleic acid sequence by hybridizing a first sequencing primer to the first adapter regions and obtaining a first read of a first portion of the target nucleic acid sequence by sequencing from the first sequencing primer, and hybridizing a second sequencing primer to the second adapter regions and obtaining a second read of a second portion of the target nucleic acid sequence by sequencing from the second sequencing primer. The first read and the second read comprise paired-end reads of the target nucleic acid sequence. Typically, sequencing from the first sequencing primer is completed before sequencing from the second primer is initiated, but in some embodiments sequencing from the first and second primers is alternated or simultaneous.

In some embodiments, the nucleic acid concatemer is produced by providing a circular nucleic acid molecule and performing rolling circle amplification using the circular nucleic acid molecule as a template to produce the nucleic acid concatemer. The circular nucleic acid molecule comprises a central region comprising the forward strand and the complementary reverse strand of the target nucleic acid sequence. The central region, which is typically double-stranded, has two ends. The forward strand is connected to the reverse strand at one end with a first connecting region, and the forward strand is connected to the reverse strand at the other end with a second connecting region. The first and second connecting regions differ from each other and are the complements of the adapter regions in the resulting concatemer. The circular nucleic acid molecule and the nucleic acid concatemer are typically but not necessarily DNA molecules.

In one class of embodiments, rolling circle amplification is performed in solution. The resulting concatemer can then be bound to a surface, e.g., within an orderly or disordered array of other concatemers produced using the methods. The location of a given concatemer within the array can be predetermined or random. Optionally, the primer extended in the rolling circle amplification reaction includes a first member of a binding pair (e.g., biotin) and the surface bears the second member of the binding pair (e.g., avidin or streptavidin). In another class of embodiments, the rolling circle amplification step is performed on the surface of a solid support. For example, the primer extended in the rolling circle amplification reaction can be bound to the surface, e.g., within an orderly or disordered array of identical primers, before or after association with the circular nucleic acid molecule and prior to initiation of the amplification reaction. The primer can be bound to the surface covalently or noncovalently (e.g., through a binding pair as noted above).

In one class of embodiments, sequencing from the first and second sequencing primers is performed at the same time. A first set of detectable signals produced by sequencing from the first sequencing primer can be distinguishable from a second set of detectable signals produced by sequencing from the second sequencing primer, e.g., on the basis of their intensity. A difference in signal intensity between the first and second sequencing processes can be produced, for example, by providing the first and second sequencing primers at different concentrations, by providing one of the sequencing primers as a mixture of extendable and non-extendable oligonucleotides, and/or by employing first and second sequencing primers that anneal to their respective adapter regions with significantly different efficiencies. Mapping to a known reference sequence can facilitate determination of the first and second reads.

Although in some embodiments sequencing from the first and second sequencing primers is alternated or simultaneous, sequencing from the first sequencing primer is more typically completed before sequencing from the second sequencing primer is initiated. In some embodiments in which sequencing from the second primer is performed after sequencing from the first primer, after the first read is obtained, the nascent strands formed by sequencing from the first sequencing primer are removed, e.g., before the second sequencing primer is hybridized to the second adapter regions on the concatemer. The nascent strands can be removed by any suitable process, e.g., cleavage and washing, exonuclease digestion, or denaturation. In other embodiments in which sequencing from the second primer is performed after sequencing from the first primer, the nascent strands formed by sequencing from the first primer are not removed. For example, the nascent strands can be blocked so that they do not interfere in sequencing from the second primer. Thus, in some embodiments, the 3′ ends of the nascent strands formed by sequencing from the first primer are blocked before sequencing from the second sequencing primer, typically before the second sequencing primer is hybridized to the second adapter regions.

Many suitable sequencing processes are known in the art and can be applied to the practice of the present methods. For example, sequencing from the first and second sequencing primers can involve a sequencing by incorporation, sequencing by ligation, or sequencing by hybridization technique. In a preferred class of embodiments, sequencing from the first and second sequencing primers comprises performing a sequencing by binding technique. During sequencing, the first and second sequencing primers are optionally extended with a strand-displacing polymerase. Sequencing can be performed in the presence of a single-stranded binding protein.

In some embodiments, e.g., in which a polymerase lacking strand displacement activity is employed, a first masking strand complementary to the forward strand is synthesized prior to sequencing from the first sequencing primer, and/or a second masking strand complementary to the reverse strand is synthesized prior to sequencing from the second sequencing primer. Typically, a first masking strand complementary to the forward strand is produced prior to hybridizing the first sequencing primer to the first adapter regions, and a second masking strand complementary to the reverse strand is produced prior to hybridizing the second sequencing primer to the second adapter regions. Alternatively, the first and second sequencing primers can be blocked at their 3′ end with a reversible terminator; the first primer can be hybridized to the first adapter region before first masking strand synthesis occurs, and the second primer can be hybridized to the second adapter region before second masking strand synthesis is performed. The reversible terminator is removed prior to initiation of sequencing from the primer.

The nucleic acid concatemer typically includes many copies of its repeating unit, e.g., at least a number of copies that will provide a detectable signal in the sequencing technique to be employed. For example, the nucleic acid concatemer can include at least 10 sequential copies of the first adapter region, the forward strand, the second adapter region, and the reverse strand, e.g., at least 50, at least 100, at least 500, at least 1000, at least 5000, or at least 10,000 sequential copies. In some embodiments, the nucleic acid concatemer includes between 50 and 20,000 sequential copies of the first adapter region, the forward strand, the second adapter region, and the reverse strand, e.g., between 50 and 10,000 or between 100 and 5,000 sequential copies. Those of skill in the art will understand that the number of copies used can vary, for example, depending on the type of analysis that is being performed. Where the length of the repeating unit is small, e.g., less than a few hundred bases, the number of copies may be higher than where the repeating unit is larger, e.g., thousands to tens of thousands of bases. One consideration is the overall molecular weight of the concatemer that is produced. Those of skill in the art will understand how to control the number of copies and overall molecular weight of the concatemer for the analysis they are carrying out.

Another general class of embodiments provides methods for nucleic acid sequencing. In the methods, a nucleic acid concatemer is provided that comprises multiple sequential copies of: a first adapter region, a forward strand of a target nucleic acid sequence, a second adapter region different from the first adapter region, and a reverse strand of the target nucleic acid sequence that is complementary to the forward strand. A sequencing process is performed to determine the sequence of at least one portion of the target nucleic acid (e.g., a portion of the forward strand, of the reverse strand, or both). In one class of embodiments, a masking primer is hybridized to the second adapter regions and extended (e.g., with a strand-displacing polymerase) to produce a first masking strand that is complementary to the forward strand. The first masking strand is not also complementary to the entirety of the first adapter region. A first sequencing primer is hybridized to the first adapter regions, and a first read of a first portion of the target nucleic acid sequence is obtained by sequencing from the first sequencing primer. Sequencing from the first sequencing primer is performed after production of the first masking strand. Synthesis of the first masking strand typically but not necessarily occurs before hybridization of the first sequencing primer to the first adapter regions.

Various strategies can be employed to ensure that the first masking strand is not complementary to the entirety of the first adapter region, by stopping extension at a suitable position. In one class of embodiments, prior to extending the masking primer, an oligonucleotide that blocks strand displacement is hybridized to the first adapter regions. In one class of embodiments, the first adapter region comprises at least one non-natural nucleotide, and the masking primer is extended under conditions that exclude the complement of the at least one non-natural nucleotide. In one class of embodiments, a nick is introduced into the first adapter region prior to extension of the masking primer. The resulting fragments can be maintained in proximity, for example, by hybridizing one end of a staple oligonucleotide to the first adapter regions and hybridizing the other end of the staple oligonucleotide to the second adapter regions. In certain embodiments, a single oligonucleotide can serve as both a sequencing primer and as a staple oligonucleotide. Thus, optionally the 5′ end of the first sequencing primer hybridizes to the second adapter regions and/or the 5′ end of the masking primer hybridizes to the first adapter regions.

Where paired-end reads from the target nucleic acid sequence are desired, any of various approaches can be employed to obtain the second read. The second read can be obtained from the other strand of the target within the concatemer. Thus, in one class of embodiments, after completion of sequencing from the first sequencing primer, the nascent strand produced by sequencing from the first sequencing primer is further extended to produce a second masking strand. The second masking strand is complementary to the reverse strand but is not complementary to the entirety of the second adapter region. The first masking strand is removed. A second sequencing primer is hybridized to the second adapter regions, and a second read of a second portion of the target nucleic acid sequence is obtained by sequencing from the second sequencing primer. In some embodiments, the masking primer comprises a 5′ phosphate group, and the first masking strand is removed by digestion with lambda exonuclease.

The second read can be obtained from a strand produced by extending the first sequencing primer rather than directly from the concatemer. Accordingly, in one class of embodiments, the first sequencing primer comprises a 5′ region and a 3′ region that each hybridize to the first adapter region and that flank a central region that does not hybridize to the first adapter region. A portion of the first adapter region remains single-stranded when the first sequencing primer is hybridized to the first adapter region. The nascent strand produced by sequencing from the first sequencing primer is further extended to produce a first extended strand complementary to the reverse strand. This first extended strand is typically not also complementary to the entirety of the second adapter region. A displacement primer is hybridized to the single-stranded portion of the first adapter region and extended with a strand-displacing polymerase to displace the first extended strand from the reverse strand. The 5′ region of the first extended strand remains hybridized to the first adapter region after displacement of the remainder of the extended strand. A second sequencing primer is hybridized to the first extended strand, and a second read of a second portion of the target nucleic acid sequence is obtained by sequencing from the second sequencing primer.

Essentially all of the features noted above apply to these embodiments as well, as relevant, e.g., with respect to number of copies of the repeating unit in the concatemer and the like.

In some embodiments, the nucleic acid concatemer is produced by providing a circular nucleic acid molecule and performing rolling circle amplification using the circular nucleic acid molecule as a template to produce the nucleic acid concatemer. The circular nucleic acid molecule comprises a central region comprising the forward strand and the complementary reverse strand of the target nucleic acid sequence. The central region, which is typically double-stranded, has two ends. The forward strand is connected to the reverse strand at one end with a first connecting region, and the forward strand is connected to the reverse strand at the other end with a second connecting region. The first and second connecting regions differ from each other and are the complements of the adapter regions in the resulting concatemer. The circular nucleic acid molecule and the nucleic acid concatemer are typically but not necessarily DNA molecules.

In one class of embodiments, rolling circle amplification is performed in solution. The resulting concatemer can then be bound to a surface, e.g., within an orderly or disordered array of other concatemers produced using the methods. The location of a given concatemer within the array can be predetermined or random. Optionally, the primer extended in the rolling circle amplification reaction includes a first member of a binding pair (e.g., biotin) and the surface bears the second member of the binding pair (e.g., avidin or streptavidin). In another class of embodiments, the rolling circle amplification step is performed on the surface of a solid support. For example, the primer extended in the rolling circle amplification reaction can be bound to the surface, e.g., within an orderly or disordered array of identical primers, before or after association with the circular nucleic acid molecule and prior to initiation of the amplification reaction. The primer can be bound to the surface covalently or noncovalently (e.g., through a binding pair as noted above).

Many suitable sequencing processes are known in the art and can be applied to the practice of the present methods. For example, sequencing from the first sequencing primer and optional second sequencing primer can involve a sequencing by incorporation, sequencing by ligation, or sequencing by hybridization technique. In a preferred class of embodiments, sequencing from the first sequencing primer and optional second sequencing primer comprises performing a sequencing by binding technique. During sequencing, the first and second sequencing primers are optionally extended with a polymerase that lacks strand displacement activity or that has weak strand displacement activity.

Compositions, systems, and kits related to, produced by, or of use in the methods are also features of the invention. For example, one general class of embodiments provides a composition that includes an array of nucleic acid concatemers. The concatemers are bound to a surface, e.g., with different concatemers at different sites in an orderly arrangement or with different concatemers at different sites at randomly distributed positions in a disordered array. The location of a given concatemer within the array can be predetermined or random. Each concatemer comprises multiple copies of: a first adapter region comprising a first sequencing primer binding site, a forward strand of a target nucleic acid sequence, a second adapter region different from the first adapter region, and a reverse strand of the target nucleic acid sequence complementary to the forward strand. The second adapter region can include a second sequencing primer binding site that differs in sequence from the first sequencing primer binding site.

The concatemers can be covalently or noncovalently bound to the surface. For example, each concatemer can comprise a first member of a binding pair (e.g., biotin) that is bound to the second member of the binding pair (e.g., avidin or streptavidin) bound in turn to the surface.

In some embodiments, a first sequencing primer is hybridized to the first sequencing primer binding sites. In some embodiments, a second sequencing primer is hybridized to the second sequencing primer binding sites. The composition can include nascent strands produced by extension of the first and/or second sequencing primer. Nascent strands produced by extension of the first sequencing primer are optionally blocked. The composition optionally also includes a polymerase (e.g., a strand-displacing polymerase or a polymerase that lacks strand displacement activity), one or more nucleotides (e.g., naturally occurring nucleotides, non-natural nucleotides, labeled nucleotides, reversible terminator nucleotides, and/or chain-termination nucleotides), a masking primer, a blocking oligonucleotide, a displacement primer, a masking strand, a displaced strand, one or more staple oligonucleotides, and/or other reagents employed in sequencing processes. The composition is optionally present in a nucleic acid sequencing system.

Essentially all of the features noted above apply to these embodiments as well, as relevant, e.g., with respect to number of concatemers in the array, number of copies of the repeating unit in the concatemer, inclusion of a nuclease in the composition for removal of nascent or masking strands, inclusion of single-stranded binding protein in the composition, suitable array substrates, and/or the like.

Another general class of embodiments provides a kit that includes a solid support configured to bind a multiplicity of nucleic acid concatemers, a first stem-loop adapter, a second stem-loop adapter different from the first stem-loop adapter, reagents for performing rolling circle amplification (e.g., a rolling circle amplification primer, a strand-displacing polymerase, and one or more nucleotides), a first sequencing primer, optionally a second sequencing primer, and reagents for performing nucleic acid sequencing (e.g., a polymerase and one or more nucleotides, typically including one, two, three, or four labeled nucleotides). Polymerases employed for rolling circle amplification and sequencing are typically different polymerases, but can in some embodiments be the same. The kit can also include additional reagents useful in producing a circular nucleic acid molecule, performing rolling circle amplification to produce a concatemer, and performing nucleic acid sequencing, including but not limited to buffered reaction solutions, a masking primer, a blocking oligonucleotide, a displacement primer, one or more staple oligonucleotides, a site-specific endonuclease, and/or an exonuclease. The kit typically also includes instructions for using the components, e.g., for producing a circular nucleic acid molecule, performing rolling circle amplification to produce a concatemer, and performing nucleic acid sequencing. Components of the kit are packaged in one or more containers.

Essentially all of the features noted above apply to these embodiments as well, as relevant, e.g., with respect to suitable array substrates, inclusion of single-stranded binding protein, and/or the like.

### BRIEF DESCRIPTION OF THE DRAWINGS

FIG. 1 schematically illustrates an embodiment of a method for preparing nucleic acid concatemers of the invention and using them for paired-end sequencing. In this embodiment, the nucleic acid segments produced in sequencing to produce the first read are removed prior to carrying out sequencing to produce the second read.

FIG. 2 schematically illustrates an embodiment of a method for preparing nucleic acid concatemers of the invention and using them for paired-end sequencing. In this embodiment, the nucleic acid segments produced in sequencing to produce the first read are blocked and remain in place while carrying out sequencing to produce the second read.

FIG. 3 schematically illustrates an embodiment of a method for preparing nucleic acid concatemers of the invention and using them for paired-end sequencing. In this embodiment, a statistical mixture of symmetric and asymmetric circular nucleic acid constructs is used to produce the nucleic acid concatemers that are sequenced.

FIG. 4 schematically illustrates preparation of a circular nucleic acid molecule and its use as a rolling circle amplification template to produce a nucleic acid concatemer useful in the methods.

FIG. 5 schematically illustrates an embodiment of a method for preparing nucleic acid concatemers of the invention and using them for nucleic acid sequencing. In this embodiment, a masking strand is synthesized to facilitate production of a sequence read.

FIGS. 6A-6B schematically illustrate an embodiment of a method for preparing nucleic acid concatemers of the invention and using them for paired-end sequencing. In this embodiment, masking strands are employed to facilitate production of the first and second reads.

FIGS. 7A-7B schematically illustrate an embodiment of a method for preparing nucleic acid concatemers of the invention and using them for paired-end sequencing. In this embodiment, the nascent strand produced during the first sequencing process is extended and displaced, and the second read is obtained from this extended strand.

Schematic figures are not necessarily to scale.

### DETAILED DESCRIPTION

The practice of the present invention may employ, unless otherwise indicated, conventional techniques and descriptions of organic chemistry, polymer technology, molecular biology (including recombinant techniques), cell biology, biochemistry, and immunology, that are within the skill of the art. Such conventional techniques include polymer array synthesis, hybridization, ligation, phage display, and detection of hybridization using a label. Specific illustrations of suitable techniques can be had by reference to the examples herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, Oligonucleotide Synthesis: A Practical Approach 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting. A variety of additional terms are defined or otherwise characterized herein.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The term “about” as used herein indicates the value of a given quantity varies by +/−10% of the value, or optionally +/−5% of the value, or in some embodiments, by +/−1% of the value so described.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

By “nucleic acid,” “polynucleotide,” “oligonucleotide,” or grammatical equivalents herein means at least two nucleotides covalently linked together. A nucleic acid of the present invention will generally contain phosphodiester bonds, although in some cases, nucleic acid analogs are included that may have alternate backbones, comprising, for example, phosphoramide, phosphorothioate, phosphorodithioate, and peptide nucleic acid (PNA) backbones and linkages. Other analog nucleic acids include those with positive backbones; non-ionic backbones, and non-ribose backbones, including those described in U.S. Pat. Nos. 5,235,033 and 5,034,506. A nucleic acid may also have other modifications, such as the inclusion of heteroatoms, the attachment of labels, such as dyes, or substitution with functional groups which will still allow for base pairing and for recognition by a polymerase or other enzyme. The term “nucleic acid” thus encompasses any physical string of monomer units that can be corresponded to a string of nucleotides, including a polymer of nucleotides (e.g., a typical DNA or RNA polymer), modified oligonucleotides (e.g., oligonucleotides comprising nucleotides that are not typical to biological RNA or DNA, such as 2′-O-methylated oligonucleotides), and the like.

The term “single-stranded” can refer, e.g., to a single polymer nucleic acid chain, or to a region within a nucleic acid polymer chain that is not base paired to a complementary region within the same chain or a different chain, depending on context. Similarly, the term “double-stranded” can refer to two polymer chains that are hybridized to each other, or to a double helical region where at least one participating chain also includes other portions that are not base paired, as will be clear from context.

A “nucleotide sequence” is a polymer of nucleotides (an oligonucleotide, a DNA, a nucleic acid, etc.) or a character string representing a nucleotide polymer, depending on context. In some cases, the term “nucleotide sequence” refers to the actual sequence of bases in a nucleic acid, and in some cases the term “nucleotide sequence” refers to a measured or determined sequence.

To “sequence” a strand of a target nucleic acid means to determine the order and identity of the nucleotides comprising that strand and/or its complement, for example, by producing a read of the strand through any of the various nucleic acid sequencing techniques known in the art. Since many such techniques employ a primer that is complementary to the strand to be sequenced (for example, a primer that is extended as the target's sequence is determined), sequencing is said to be performed “from” the primer.

A “target nucleic acid sequence” is a nucleic acid or region thereof whose nucleotide sequence, or at least one portion thereof, is to be determined. Since, as is well known in the art, a particular nucleic acid sequence encodes the complementary sequence, any target nucleic acid sequence can be expressed as a “forward” sequence (or strand) or as its complementary “reverse” sequence (or strand). Certain methods of the invention determine a portion of the nucleotide sequence of the forward strand and a portion of the nucleotide sequence of the reverse strand (typically, a different portion); since the two strands are complementary, this process typically determines the nucleotide sequence of two portions of the forward strand and two portions of the reverse strand (generally, the two opposite ends of each). The terms “forward” and “reverse” are used herein in a relative rather than an absolute sense to convey that one strand or sequence is the complement of the other; no implication of function (e.g., coding versus noncoding), position within a chromosome, order of amplification, or other such information is intended.

A “read” of a nucleic acid sequence is a measured or inferred sequence of nucleotides or base pairs (or of nucleotide or base pair probabilities). A read can correspond to the entirety of or to a portion of a single DNA fragment. A read typically provides the measured or inferred order and identity of bases in a region of DNA, and a read can also include other information, such as quality scores, probabilities, etc.

Oligonucleotides employed in the present disclosure that are designed to anneal specifically to a sufficiently complementary nucleic acid sequence in a target nucleic acid under appropriately stringent hybridization conditions are sometimes referred to herein as “primers,” while the sequence to which they anneal is referred to as the “primer binding site” (or “priming site”). Thus, a primer that is “specific for” a primer binding site in a nucleic acid, e.g., a primer binding site in an adapter region, is one that includes a nucleic acid sequence that hybridizes preferentially to the primer binding site under appropriately stringent hybridization conditions. Appropriately stringent hybridization conditions are regularly determined by the ordinarily skilled person in the art. The nucleic acid sequence in a primer that hybridizes to the primer binding site is sometimes referred to herein as the “primer region” or similar phrase. Primers often include regions that do not participate directly in hybridizing to the primer binding site and that may have a particular use in methods described herein. Primers can be used for different functions, including but not limited to nucleic acid synthesis and/or capture of nucleic acids containing their cognate primer binding site. For example, a “capture primer” (or “capture oligonucleotide”) can be used to isolate nucleic acids that contain the specific capture primer binding site, e.g., present in an adapter or adapter region, and often include a first member of a binding pair (e.g., a nucleic acid sequence, biotin, avidin, antigen, antibody or binding fragment thereof, etc.) or are attached directly to a solid support. As another example, a “synthesis primer” is one that can be used to prime nucleic acid synthesis by a nucleic acid polymerase (under nucleic acid synthesis conditions) and, in certain specific applications, can be used as a sequencing primer in sequencing by synthesis (SBS) or sequencing by binding (SBB) applications or as a primer in a nucleic acid amplification reaction (e.g., a rolling circle amplification reaction).

As used herein, a “substantially identical” nucleic acid is one that has at least 80%, 85%, 90%, 95%, 96%, 97%, 98% or 99% sequence identity to a reference nucleic acid sequence. The length of comparison is preferably the full length of the nucleic acid, but is generally at least 20 nucleotides, 30 nucleotides, 40 nucleotides, 50 nucleotides, 75 nucleotides, 100 nucleotides, 125 nucleotides, or more.

By “asymmetric nucleic acid” is meant a nucleic acid that has a different nucleic acid composition at the first end of the nucleic acid as compared to the second end of the nucleic acid. An asymmetric nucleic acid that is particularly useful in the context of the invention is asymmetrically-tagged, i.e., having a first adapter attached to a first end and a second adapter attached to a second end where the first adapter and the second adapter have at least one nucleic acid composition difference. The nucleic acid composition difference between the first adapter and the second adapter can be any desired difference, including but not limited to one or more nucleic acid sequence difference (e.g., substitutions, deletions, insertions, inversions, rearrangements (e.g., a different order of functional domains), or any combination thereof). In certain embodiments, and as detailed herein, asymmetric nucleic acids include a primer binding site for an amplification primer at one end but not at the second end.

The term “nucleic acid concatemer” as used herein refers to a continuous nucleic acid polymer chain (e.g., a single DNA chain) that contains multiple copies of the same nucleic acid sequence linked in series. Concatemers particularly useful in the context of the present invention include multiple tandem copies of a sequence that includes: a first adapter region, one strand of a target nucleic acid sequence, another adapter region different from the first, and the complementary strand of the target nucleic acid sequence. An “adapter region” is a nucleic acid sequence that connects the 3′ end of a given strand of a target nucleic acid sequence with the 5′ end of the other strand of the target nucleic acid sequence within a concatemer. In embodiments in which stem-loop adapters are ligated to the ends of a double-stranded fragment to form a circular nucleic acid from which the concatemer is produced, e.g., by rolling circle amplification, the adapter regions are complementary to sequences contributed by the adapters (i.e., each adapter region is complementary to one of the “connecting regions” in the circular nucleic acid that connect the 3′ end of one strand of the target nucleic acid sequence with the 5′ end of the other strand of the target nucleic acid sequence within the circular nucleic acid).

By “binding pair” is meant any two moieties that specifically bind to each other under at least one binding condition. Members of binding pairs include, but are not limited to, complementary single-stranded nucleic acid sequences, biotin/avidin or biotin/streptavidin (as well as biotin/neutravidin, biotin/traptavidin, and the like), antigen/antibody, hapten/antibody (e.g., digoxigenin/anti-digoxigenin antibody), ligand/receptor, etc. (It is noted that the antigen/hapten binding fragment of an antibody may be employed rather than the entire antibody.)

By “linker” is meant a moiety that functions to attach a first functional element or moiety to another. With respect to attaching nucleic acid domains to each other or to distinct moieties, linkers could be additional nucleotide residues (DNA, RNA, PNA, etc.), peptides, carbon-chain, poly-ethylene-glycol spacers, etc. Attachment of functional elements/moieties with a linker can be covalent or non-covalent. No limitation in this regard is intended.

By “strand-displacing nucleic acid polymerase,” “strand-displacing polymerase,” and equivalents thereof is meant a nucleic acid polymerase that has both 5′ to 3′ template dependent nucleic acid synthesis activity and 5′ to 3′ strand displacement activity. Thus, when such a polymerase encounters a double-stranded region of a template during nucleic acid synthesis, it will displace the non-template strand while continuing nucleic acid synthesis on the template strand. On circular templates (e.g., templates having a double-stranded insert with hairpin adapters at both ends, as shown in the figures), such polymerases can enter into rolling circle replication under suitable nucleic acid synthesis conditions. While any suitable strand-displacing nucleic acid polymerase can be used, in certain embodiments the polymerase is a phi29 (Φ29) DNA polymerase or a modified version thereof. Where a modified recombinant D29 DNA polymerase is employed, it can be homologous to a wild-type or exonuclease deficient Φ29 DNA polymerase, e.g., as described in U.S. Pat. Nos. 5,001,050, 5,198,543, or 5,576,204, the full disclosures of which are incorporated herein by reference in their entirety for all purposes. Alternately, the modified recombinant DNA polymerase can be homologous to other Φ29-type DNA polymerases, such as B103, GA-1, PZA, Φ15, BS32, M2Y, Nf, G1, Cp-1, PRD1, PZE, SFS, Cp-5, Cp-7, PR4, PR5, PR722, L17, Φ21, or the like. For nomenclature, see also, Meijer et al. (2001) “Φ29 Family of Phages” Microbiology and Molecular Biology Reviews, 65(2):261-287. Exemplary suitable polymerases are described, for example, in U.S. Pat. Nos. 8,420,366 and 8,257,954, both entitled “Generation of modified polymerases for improved accuracy in single molecule sequencing,” U.S. patent application publication nos. 2007-0196846, 2008-0108082, 2010-0075332, 2010-0093555, 2012-0034602, 2013-0217007, 2014-0094374, and 2014-0094375, and International Patent Application Nos. WO 2007/075987, WO 2007/075873, WO 2007/076057, incorporated herein by reference in their entirety for all purposes. Many additional suitable strand-displacing polymerases are known in the art or commercially available.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### Methods of Paired-End Sequencing

The present disclosure is generally directed to improved methods for nucleic acid sequencing, particularly improved methods for carrying out paired-end sequencing. Paired-end, or pairwise, sequencing typically involves determining the nucleotide sequence of a first region and a second region of a target sequence of interest (e.g., determining the sequence of the two opposite ends of a nucleic acid fragment). In some embodiments, the first region and second region that are sequenced are separated by a known or unknown nucleic acid segment that is not sequenced. In other embodiments, the first region and the second region are overlapping, and the sequence of the full-length target region can be determined.

In some aspects, the invention provides for producing sequencing templates and carrying out paired-end sequencing on those sequencing templates. The sequencing templates can be single nucleic acid chains that each have the repeating structure: adapter region 1, forward nucleic acid strand of the target, adapter region 2, reverse nucleic acid strand of the target. These nucleic acids are concatemeric molecules. The repeating structure can be present, for example, hundreds to thousands of times in the concatemer. The concatemer is single-stranded in the sense that it is a single polymer chain; it will be evident that under some conditions this chain can have secondary structure, e.g., where self-complementary portions of the chain (e.g., the forward and reverse regions) base pair to form double-stranded regions separated by regions that remain unpaired.

The concatemeric nucleic acid sequencing templates for use in the invention can be produced by rolling circle amplification of a molecule having a central double-stranded region that is connected through a single-stranded loop on each of its ends to form a circular molecule. These types of topologically circular molecules are sometimes referred to, as described herein, as SMRTBELLs®, and can be produced, e.g., by ligating two different hairpin (i.e., stem-loop) adapters to the two ends of a double-stranded DNA fragment. The hairpin regions at either end of the central self-complementary double-stranded region (e.g., the regions contributed by the hairpin adapters) can be called connector regions. For the instant invention, the molecules preferably have a different connector on one end of the central region than on the other end. In other words, the molecules are asymmetric. The molecules are asymmetric such that they form preferred sequencing templates of the invention in which adapter region 1 is different than adapter region 2 after rolling circle amplification of the circular molecule, e.g., using an amplification primer complementary to one of the two connector regions. Concatemeric nucleic acid sequencing templates can also be produced by other techniques known in the art, for example, by multiple displacement amplification (MDA) using random or construct-specific primers. For example, a concatemer produced by rolling circle amplification of an asymmetric circular nucleic acid can be used in a multiple displacement amplification reaction with primers specific for the adapter-derived regions of the concatemer to produce concatemers useful as sequencing templates.

The invention provides methods of sequencing of concatemeric templates to obtain paired-end reads of a target sequence of interest. Sequencing can be carried out in any suitable manner. The sequencing method can be a stepwise sequencing method. The sequencing method can be, for example, sequencing by synthesis (SBS), sequencing by binding (SBB), or sequencing by ligation. The concatemeric structure provides multiple sites for carrying out stepwise sequencing, since the concatemeric templates of the invention include clonal populations of the target nucleic acid sequences. An individual concatemeric molecule has clonal populations of both the forward strand and the reverse strand of a particular target region of interest. The sequencing can be carried out by first introducing a sequencing primer that hybridizes to adapter region 1. Stepwise sequencing from this primer provides a sequence read of the first strand. The sequencing process produces a nascent nucleic acid strand that is complementary to the first strand. This sequencing process can proceed for the desired number of nucleotides. Typically, the read includes a portion of the first strand's sequence, but it can include the entire length of the first strand. After this sequencing of the first strand, a second sequencing primer is introduced. The second primer hybridizes to adapter region 2, and stepwise sequencing is carried out to provide a sequencing read of part or all of the complementary second strand. Because of the topology of the sequencing template, the first read corresponds to reading from a first end of the target sequence, and the second read corresponds to reading from the second end of the target sequence, providing a pair-wise, or paired-end, sequence of the target sequence of interest. If the first read is of one end of a “sense” strand of the target nucleic acid, then the second read will be of the opposite end of the “antisense” strand of the target nucleic acid (and vice versa). Base complementarity rules can be used to convert a sequence or sequence region to its complement for sequence analysis. Such analysis can employ any desired combination of sequences, e.g., the first and second reads, the complements of the first and second reads, or one read and the complement of the other read, since all of these provide equivalent information.

When carrying out the second sequencing process to produce the second read, confounding signal from the extended primer of the first sequencing process is undesirable. Thus, in some cases, the first primer and nascent strand produced in the first sequencing process are removed or blocked prior to carrying out the second sequencing process.

The nascent strands from the first sequencing process can be removed by any suitable process. For example, they can be degraded or they can be removed by denaturation. Degradation can be carried out enzymatically, e.g. by exonuclease treatment. In one example, the 5′ end of the concatemer is protected (e.g., by a biotin or bis-biotin moiety through which the concatemer is bound to a surface) and a 5′ to 3′ exonuclease is employed to remove the nascent strands. As another example, digestion with a 3′ to 5′ exonuclease can be employed to remove the nascent strands; digestion time is typically limited such that the short nascent strands are removed. While a small portion of the concatemer may also be removed during this time, typically the concatemer includes so many copies of the repeating unit that the removal of a small portion has little to no effect on the subsequent sequencing process. The nascent strands can be cleaved, e.g., by endonuclease treatment, into shorter pieces that can be washed away. Degradation can also be accomplished by including nucleotides subject to subsequent cleavage, e.g., by incorporating uracil residues into the nascent strand during sequencing and employing a mixture of uracil DNA glycosylase (UDG) and DNA glycosylase-lyase endonuclease VIII, endonuclease IV, or APE1 for cleavage, optionally followed by washing to remove the resulting pieces. Denaturation can be carried out by changing any suitable conditions such as ionic strength and/or temperature. Combinations of degradation and denaturation can be employed.

In some cases, the primer and nascent strand from the first sequencing process is blocked from further sequencing prior to carrying out the second sequencing process. There are many methods of blocking of a DNA strand at the 3′ end to keep it from generating signal that will confound the second sequencing process. For example, a dideoxynucleotide, a 3′-blocked nucleotide (e.g., a 3′-O-azido dNTP), or another group that blocks 3′ extension can be incorporated; many such groups are known in the art. See, e.g., U.S. patent application publication US2020/0032322, which describes ternary complex inhibitor moieties useful for blocking the 3′ end of the nascent strand and which is hereby incorporated by reference herein in its entirety for all purposes. The 3′ blocking groups can be referred to as terminators. In some cases, reversible terminators can be used. Examples of suitable terminators are provided, for example, in Chen et al. (2013) “The history and advances of reversible terminators used in new generations of sequencing technology” Genomics Proteomics Bioinformatics 11(1):34-40. In some cases, the blocking can be irreversible. The blocking can be tailored to the type of sequencing that is being carried out. For example, if the sequencing method used in the second sequencing process is sequencing by binding, it can be desirable to provide a blocking group that not only prevents extension, but also prevents cognate nucleotide binding that could provide a false background signal.

The methods of the invention are optionally used for highly multiplexed sequencing. The concatemeric sequencing templates are typically immobilized, for example, to a substrate. The substrate will have many concatemeric sequencing templates which are individually resolvable. In some cases, optical detection of the sequencing reaction is used. Other detection methods, for example, electronic detection methods can be used. The number of sequencing templates can be chosen for a particular application. In some cases, millions to billions of concatemeric sequencing templates can be sequenced in parallel on the same substrate (e.g., at least one million, at least ten million, at least 100 million, at least one billion, at least two billion, or at least three billion concatemeric templates). Typically, for such parallel sequencing, each concatemeric sequencing template will have the same first adapter regions and second adapter regions as do the other the concatemeric sequencing templates, while the forward and reverse regions in different concatemeric sequencing templates will represent different target molecules. A library of such different target molecules can be produced, for example, by fragmenting DNA from an organism of interest. Many methods of library formation are known in the art. Since different concatemers include the same adapter regions, a single pair of primers can be employed in sequencing the different targets.

In describing aspects of the methods disclosed herein, reference will be made to the figures. It is to be understood that the figures merely illustrate specific embodiments of the disclosed methods and are not intended to be limiting.

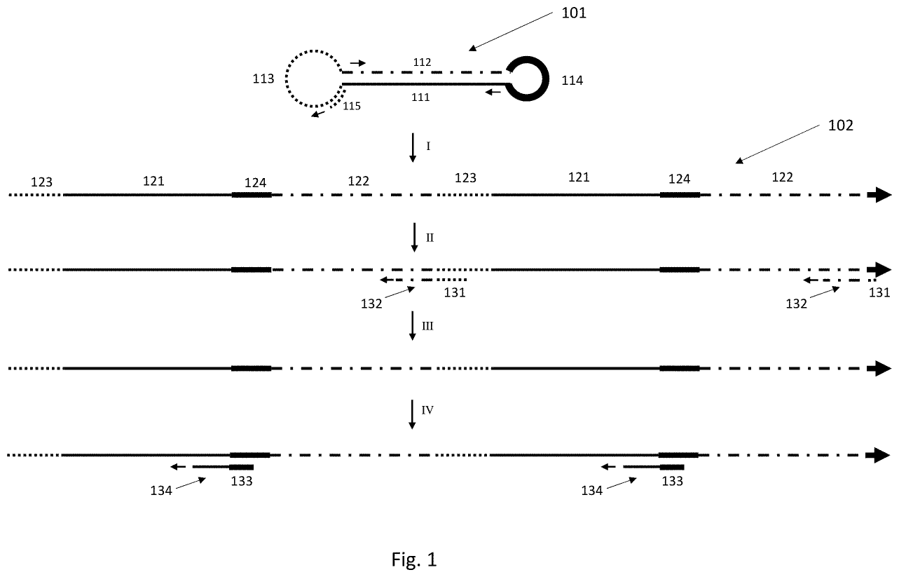

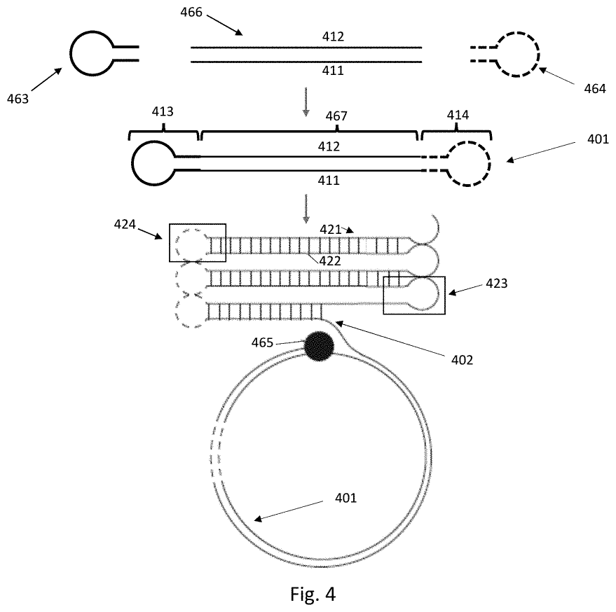

FIG. 1 schematically illustrates an embodiment of a method of the invention. An asymmetric rolling circle amplification template 101 is provided. Circular template 101 includes a central target region including self-complementary forward (111) and reverse (112) regions. The template is capped on one end with first connecting region 113 and capped on the other end with second connecting region 114. First connecting region 113 has a different nucleic acid sequence than second connecting region 114. The connecting regions can have sequences with various functions, such as barcodes, priming sites, restriction sites, recognition sites, etc. Note that that while the connectors or connecting regions are shown as corresponding to the single-stranded loop regions of template 101, in many cases, the connectors will also have self-complementary regions that extend into the double-stranded portion of circular nucleic acid 101, e.g., where hairpin adapters each having a single-stranded loop and double-stranded stem are ligated to a double-stranded fragment to form the circular nucleic acid. (See, e.g., the example illustrated in FIG. 4.) One or more priming sites, barcodes, and other useful sequences (or the complement thereof) can be included in the single-stranded or double-stranded portion (or both) of such adapters and therefore in the resulting connecting regions. One of the two connectors (113, in this example) will have a rolling circle amplification initiation site such as a rolling circle amplification primer binding site, to which primer 115 is hybridized. The amplification primer binding site is typically, but not necessarily, located in a single-stranded loop region of the connecting region.

Here, in step I, rolling circle amplification primer 115 is extended with a strand-displacing polymerase such as phi29. The polymerase proceeds repeatedly around circular template 101 in the direction indicated by the small arrows, forming concatemer 102 with the repeating structure: first adapter region 123-forward strand 121-second adapter region 124-reverse strand 122 (reading from 5′ to 3′). The figure shows two copies of the unit of the concatemer. The arrow at the end of concatemer 102 indicates that there are typically many more copies of the unit, for example, hundreds to thousands. The number of copies can be any suitable number, for example tens, hundreds, thousands, or tens of thousands of copies. Here, first adapter region 123 is the complement of first connecting region 113, forward strand 121 is the complement of reverse strand 112 in rolling circle template 101 and is thus substantially identical in sequence to forward strand 111 of 101, second adapter region 124 is the complement of second connecting region 114, and reverse strand 122 is the complement of forward strand 111 in rolling circle template 101 and is thus substantially identical in sequence to reverse strand 112 of 101. While the target segments are shown as directly connected to segments complementary to the single-stranded loop regions of the circular nucleic acid, it is understood that there can be, and typically will be, other intervening sequences between these segments. For example, where stem-loop adapters are used to construct template 101, the complement of one strand of the stem of the adapter would be present between the complement of the loop and the forward target region in the resulting concatemer. As described above for the rolling circle template, these intervening sequences can be used structurally (e.g. self-complementary regions), or the intervening sequences can have specific functions, e.g. cut sites, primer binding sites, recognition sites, or barcodes including molecular barcodes or unique molecular identifiers (UMIs).

This rolling circle amplification (RCA) process can be carried out in solution or can be carried out by extending an RCA primer that is bound to a substrate. Where the RCA primer is bound to a substrate, the result is a concatemer that is bound to the substrate through the primer. The primer can be bound covalently or non-covalently to the surface, e.g., as noted hereinbelow or using techniques well known in the art. If the RCA process is carried out in solution, the concatemers produced can be deposited and immobilized onto a substrate, e.g., as noted hereinbelow or using techniques well known in the art.

Step II shows the first sequencing process, which uses first primer (131, dotted line) that hybridizes to a primer binding site in first adapter region 123. This primer binding site on first adapter region 123 is the complement of a sequence that is included in first connecting region 113 but that is not present in second connecting region 114. The sequencing process can be carried out on a concatemer or a plurality of concatemers immobilized on a substrate. The sequencing process can be by a stepwise sequencing process such as sequencing by synthesis (SBS), sequencing by binding (SBB), or sequencing by ligation. In the exemplary embodiment illustrated in FIG. 1, first sequencing primer 131 is extended to form nascent strand 132, with extension proceeding in the direction indicated by the small arrow. The sequencing process proceeds from sequencing primer 131 into target sequence 122 until the first sequencing process is halted. This process produces the first read.

In this embodiment of the method, in step III, nascent strands 132 produced by extension of primer 131 produced in the first sequencing process are removed. As described hereinabove, the removal can be by, e.g., denaturation, degradation (e.g., by exonuclease), or a combination of such techniques.

In step IV, the second sequencing process is carried out from sequencing primer 133 (thick line) that hybridizes to a primer binding site in second adapter region 124. This primer binding site on second adapter region 124 is the complement of a sequence that is included in second connector 114 and that is not present in first connector 113. The second sequencing process is typically carried out in the same manner as the first sequencing process, but could be carried out with a different process. In the exemplary embodiment illustrated in FIG. 1, second sequencing primer 133 is extended to form nascent strand 134, with extension proceeding in the direction indicated by the small arrow. The second sequencing process is carried out until the process is halted. It produces the second read. The second read is from a strand that is complementary to the strand from which the first read is obtained. The second read extends in from the opposite end of the target nucleic acid of interest than the first read. The combination of the first read and the second read provides a paired-end sequence of the target sequence of interest.

The length of the target sequence of interest will vary depending on the application. One example would be with a library of DNA target fragments from an organism of interest. Asymmetric hairpins are added to the ends of the target fragments to produce a library of asymmetric rolling circle amplification templates. One of the hairpins has an RCA primer binding site. Rolling circle amplification is carried out on the library to form a set of concatemeric sequencing templates of the invention. The RCA can be carried out in solution, and the set of concatemeric sequencing templates is then immobilized onto a substrate, or the RCA can extend surface bound primers. The set of concatemeric sequencing templates is immobilized onto the surface such that at least a subset of the immobilized sequencing templates are individually resolvable. The first sequencing processes and second sequencing processes are carried out on the set of individually resolvable sequencing templates using a stepwise sequencing method as described herein to produce a first read and a second read.

In some embodiments, the first and second reads do not overlap. As one example, the target fragments can be about 500 bases long. Each of the first and second sequencing processes can extend, for example, 150 bases. For the sequencing templates with target regions about 500 bases long, the first 150 bases and last 150 bases of the target would be identified, and the central region of 200 bases would not be identified. In some embodiments, e.g., for shorter target regions, there can be overlap between the first read and the second read. For example, if the target fragments are 200 bases long, and the first read and second read are each 150 bases, there will be a 100 base section in the middle of the target that is sequenced twice, providing improved accuracy within this region.

The binding sites for the first and second sequencing primers are typically, but not necessarily, located in a single-stranded loop region of their respective adapter regions. The binding sites for the sequencing primers are optionally positioned as close to the 5′ end of the adapter region as possible (e.g., as close as possible to the 5′ end of the single-stranded loop portion of the adapter region), such that sequencing proceeds into the target region as early in the process as convenient. Optionally, a barcode or other sequence tag is located between the 5′ end of the adapter region and the sequencing primer binding site such that sequence information from the tag as well as the target region is produced. One of the sequencing primer binding sites can, but need not, overlap with the complement of the rolling circle amplification primer binding site.

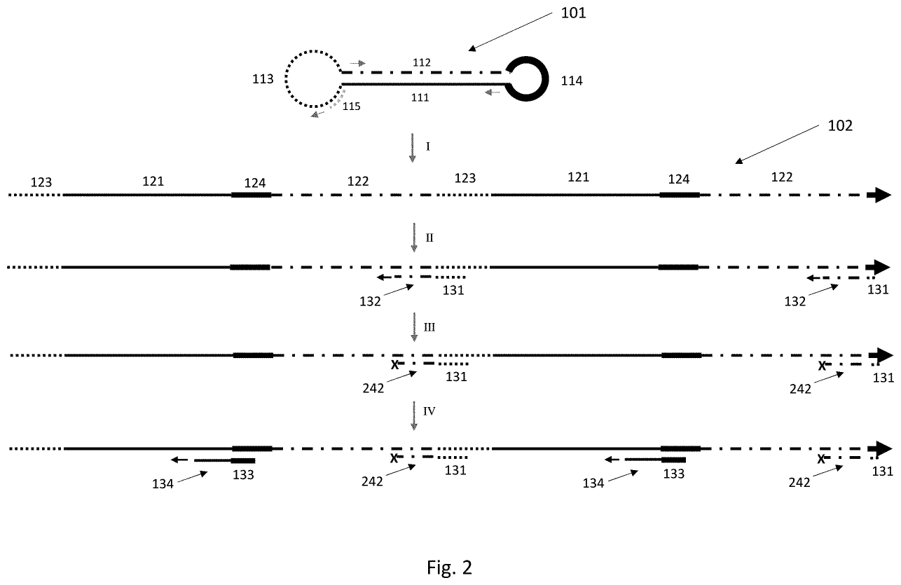

FIG. 2 schematically illustrates another embodiment of a method of the invention. The steps are similar to those recited describing FIG. 1, but here rather than removing nascent strand 132 from the first sequencing process, nascent strand 132 is blocked from providing further sequencing signal, allowing the second sequencing process to proceed without the removal of this strand. Steps I and II proceed basically as described above for FIG. 1. In step III, after the first sequencing process extending from primer 131 into reverse strand 122 is halted, a blocking agent, X, is added which prevents blocked nascent strand 242 from giving a false background signal during the subsequent second sequencing process. Step IV proceeds basically as described above for FIG. 1, except that blocked nascent strands 242 remain in place during the second sequencing process. There are many ways known in the art for blocking a nascent strand in this manner There are irreversible blocking agents that will, for example, react with the 3′ hydroxyl to prevent further extension. A dideoxynucleotide, a 3′-blocked nucleotide (e.g., a 3′-O-azido dNTP), or another group that blocks 3′ extension can be incorporated to terminate and block the nascent strands. Binding agents, e.g. binding proteins or antibodies can be used to prevent extension. In some cases, the sequencing polymerase can be disabled in a way that prevents its dissociation and halts extension, such that it acts as a blocking agent. While in many embodiments an irreversible blocking agent is employed, in other embodiments the blocking agent can be a reversible blocking agent. As noted above, examples of suitable reversible terminators are provided, e.g., in Chen et al., supra. For processes such as SBB, in addition to preventing extension, it is useful for the blocking agent to also prevent a labeled nucleotide analog from sampling the end of the blocked nascent strand and creating background signal. This can be accomplished, for example, with a binding moiety or with a bulky termination group. See, e.g., U.S. patent application publication US2020/0032322 (hereby incorporated by reference herein in its entirety for all purposes), which describes ternary complex inhibitor moieties useful for blocking the 3′ end of the nascent strand and, by inhibiting ternary complex formation, reducing background signal during the examination phase of a sequencing by binding technique.

Typically, first one sequencing process is performed from the first primer to produce a first read, and then a second sequencing process from the second primer is carried out to produce a second read. In some cases, however, one can alternate between reading from the first primer and second primer. For example, different reversible blockers can be used for the first sequencing process and second sequencing process such that either process can be stopped and started as desired. The method can be used, for example, to produce a first read and a second read, where each read is determined over several sequencing processes rather than a single sequencing process. Alternation between sequencing from the first primer and from the second primer can be occur as often as every nucleotide, if desired. In other cases, sequencing from the first and second primers can be performed simultaneously.

In some embodiments in which sequencing from the first and second sequencing primers is performed at the same time, signals detected during the sequencing process (e.g., optical signals observed from binding of fluorescently labeled cognate nucleotide analogs during SBB reactions) are distinguished on the basis of their intensity. For example, signals produced from sequencing from the first primer can be more intense than are signals produced from sequencing from the second sequencing primer (or vice versa). This can be accomplished, for example, by providing the first and second sequencing primers at different concentrations, by providing one of the primers as a mixture of extendable and non-extendable oligonucleotides (thus effectively lowering the concentration of that primer that can participate in the sequencing reaction), and/or by employing first and second primers that anneal to their respective adapter regions with significantly different efficiencies (e.g., through choice of primer sequence, inclusion of 2′-O-methyl nucleotides or other modifications in a primer that result in tighter or weaker primer binding, and/or by varying the size of the single-stranded loop in the adapter region, with a smaller loop generally reducing primer binding efficiency). As the identity of nucleotides in the first and second reads is determined from the signals observed during the sequencing process, determination of whether a particular signal was generated by sequencing from the first primer or the second primer (and therefore whether a particular nucleotide should be assigned to the first or second read) can be facilitated by mapping to a known reference sequence. For example, a set of possible sequence reads can be generated based on the observed signals and compared to a known reference sequence to obtain the reads. In a stepwise sequencing process, the nucleotide occupying each position can be identified in turn. Where the forward and reverse strands are being sequenced at the same time, two nucleotides are identified for each position (or one nucleotide, if the same nucleotide is in the next position in both strands). For a read of length n, at most 2′ possible sequences are thus generated for comparison to the reference. Such mapping can be useful in embodiments in which observed intensities differ between the first and second sequencing processes, as well as embodiments in which no intensity difference is employed and assignment of bases to the first or second read is determined by mapping rather than by a combination of mapping and observed intensity.

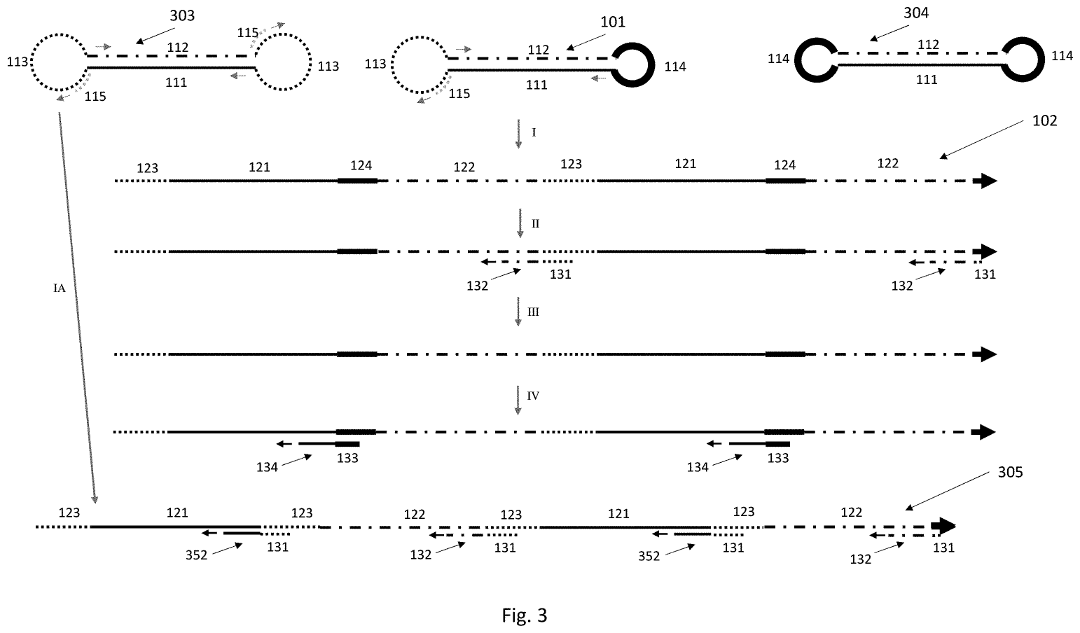

FIG. 3 shows how the paired-end sequencing process of the invention can be used with a statistical mixture of symmetric and asymmetric rolling circle amplification templates. In some cases, it is desired to use a simplified process for forming asymmetric circular RCA templates that results in the formation of both symmetric and asymmetric molecules. There are a number of ways to produce this statistical mixture that are known in the art. One approach is to start with a target DNA fragment or library of target DNA fragments, and to ligate a mixture of two different hairpin adapters to the ends. When this is done, a statistical mixture of products results. For example, if one hairpin adapter comprises connecting region 113 and the other hairpin adapter comprises connecting region 114, a mixture of about 50% asymmetric circular nucleic acid 101 having both connectors 113 and 114 and about 50% symmetric circular nucleic acids (303 having two copies of connecting region 113, and 304 having two copies of connecting region 114) would be formed when approximately equal amounts of the two adapters are provided. FIG. 3 shows these constructs. While there will be a reduction in yield from using this approach, employing such a statistical mixture can be useful, for example, where the process of obtaining high percentages of asymmetric constructs is complex, inconvenient, undesirable, or unnecessary.

FIG. 3 shows that asymmetric rolling circle amplification template 101 works just as described above. In the exemplary embodiment shown in this figure, removal of the nascent strand resulting from the first sequencing process is shown. Other methods described herein will also work in this context, e.g., for blocking instead of removal of the nascent strands. In the embodiment shown in FIG. 3, connecting region 113 has a rolling circle amplification priming site, and connecting region 114 does not. Thus, symmetric construct 304 that contains only connecting region 114 will not undergo rolling circle amplification, and therefore will not produce a concatemeric sequencing template. Symmetric constructs 303 have connecting region 113 and thus RCA priming sites on both ends. These constructs can undergo RCA amplification as shown in step IA to produce concatemer 305. Because concatemer 305 is produced from symmetric construct 303, it includes only first adapter regions 123. It thus includes a binding site for first sequencing primer 131 that extends into reverse region 122, and it also includes a binding site for first sequencing primer 131 that extends into forward region 121. Therefore, when carrying out the first sequencing process on concatemer 305, there will be a convoluted signal; extension of first sequencing primers 131 will be adding bases sequentially into two different sequences (nascent strands 132 and 352) simultaneously, so use of the information from this concatemer will be difficult to impossible for providing sequence information without the use of additional information, as discussed below. When a second sequencing process is carried out, there will be no signal from concatemeric sequencing template 305, as it does not contain a binding site for second sequencing primer 133. While these concatemeric templates will not generate a useful signal in the absence of additional manipulation, it is straightforward to identify these templates by their characteristics (e.g., convoluted signal from the first sequencing process and/or the absence of signal from the second sequencing process) and remove them from the analyses. Thus, while the presence of the symmetric RCA templates will affect yield of desirable concatemers and useable sequence data, useful paired-end sequencing data will nonetheless be obtained from those concatemeric templates 102 produced from asymmetric constructs 101. In some cases, however, the convoluted signals from templates such as 305 that are produced from symmetric constructs can be employed in sequence determination. Mapping to a known reference sequence as detailed above can be employed to obtain the first and second reads (e.g., from a set of possible reads that includes all possible combinations of the one or two nucleotides identified at each position during a stepwise sequencing process).

It will be evident that, while use of asymmetric constructs to achieve paired-end sequencing as detailed herein is preferable in many circumstances, in some instances use of asymmetric constructs is not required. For example, where a reference sequence is available (e.g., in targeted sequencing approaches where the target or pool of target nucleic acid sequences represents a particular region or regions of interest), symmetric constructs can be employed. In such embodiments, a symmetric construct is produced, rolling circle amplification is performed to produce a concatemer similar to 305, and sequencing proceeds from a single primer complementary to the single adapter region that is present in the concatemer. Mapping to known reference sequence(s) can then produce the desired sequence reads.

While symmetric constructs or mixtures of symmetric and asymmetric constructs can be employed in certain embodiments, in many embodiments asymmetric constructs are preferred. A suitable method for preparation of an asymmetric nucleic acid for use as a rolling circle amplification template in the methods of the invention is schematically illustrated in FIG. 4. To produce circular nucleic acid molecule 401, two different stem-loop adapters (463 and 464) are provided and ligated onto opposite ends of double-stranded target nucleic acid fragment 466 that includes forward strand 411 and reverse strand 412. Asymmetric circular nucleic acid 401 thus includes first connecting region 413 (contributed by adapter 463) and second connecting region 414 (contributed by adapter 464) that connect the ends of forward and reverse target strands 411 and 412 to each other. In this example, central region 467 (which includes forward and reverse strands 411 and 412 of the target nucleic acid sequence) is initially double-stranded. Connecting regions 413 and 414 each include a single-stranded loop and also a double-stranded portion that is adjacent to the central target region. As detailed above, various useful sequences (or the complements thereof) can be included in the connecting regions, such as primer binding sites, barcodes or unique molecular identifiers (UMIs), restriction sites, recognition sites, and the like.

In this example, a rolling circle amplification primer is hybridized to a primer binding site in connecting region 413. Extension of the primer by strand-displacing polymerase 465 shifts template 401 into an open circular form and produces nucleic acid concatemer 402, as shown in FIG. 4. Concatemer 402 includes multiple sequential copies of: first adapter region 423 (which is complementary to first connecting region 413), forward strand 421 of the target nucleic acid sequence (which is complementary to reverse strand 412 of circular template 401), second adapter region 424 (which is complementary to second connecting region 414 and which is therefore different from first adapter region 423, since connecting regions 414 and 413 are different from each other), and reverse strand 422 of the target nucleic acid sequence (which is complementary to forward strand 411 of 401 as well as to forward strand 421).

In the exemplary embodiment illustrated in FIG. 4, the rolling circle amplification primer is hybridized to a primer binding site in connecting region 413. A portion of first adapter region 423 (whose size depends on where in the single-stranded loop of connecting region 413 the rolling circle amplification primer binding site is located) followed by forward strand 421 thus appears at the 5′ end of the resulting concatemer. It will be evident that the primer binding site could instead be located in connecting region 414. In such an embodiment, a portion of second adapter region 424 followed by reverse strand 422 appear at the 5′ end of the resulting concatemer. Since these are immediately followed by first adapter region 423, forward strand 421, second adapter region 424, reverse strand 422, and so on, it will be evident that the two concatemers can be viewed as containing the same internal repeating unit. That is, regardless of which connecting region the rolling circle amplification primer binding site is designed into (and therefore which target strand is produced first), the resulting concatemer still includes multiple sequential copies of a repeating unit that includes the first adapter region, the forward strand, the second adapter region, and the reverse strand. As noted above, designation of target strands as forward and reverse is purely for convenience in referring to the two complementary strands. No limitation as to which strand is amplified first is intended thereby. Designation of adapter regions, connecting regions, and the like as first and second is similarly purely for convenience in referring to these elements.

The concatemer can be immobilized on a surface, e.g., at a resolvable spot or position within an orderly array or a disordered distribution of such concatemers, e.g., on a slide, chip, surface of a flow cell, or other suitable substrate. The position of a given concatemer in the orderly or disordered array can be predetermined or random. The concatemer can be immobilized, for example, on a planar surface of a substrate, on a non-planar surface of a substrate, or in a three dimensional manner within a substrate, such as within a gel matrix. The immobilization onto a substrate can be carried out in any suitable manner. In some embodiments, the concatemer is produced on the surface. For example, a rolling circle amplification primer can be immobilized on a surface, e.g., covalently, through binding of a biotin or bis-biotin group on the primer to a surface-bound biotin or bis-biotin via avidin or streptavidin, through hybridization to a complementary oligonucleotide that is immobilized on the surface, or through any of the other techniques known in the art for creating arrays of or otherwise immobilizing oligonucleotides. (See, e.g., U.S. Pat. No. 6,274,320, which describes techniques for creating arrays of attachment sites on a solid support and attaching primers thereto.) Extension of the primer then results in a concatemer immobilized at that position. In other embodiments, the concatemer is produced in solution and then immobilized through any of the techniques known in the art for creating arrays of or otherwise immobilizing nucleic acids. For example, a biotinylated rolling circle amplification primer can be extended in solution, and the resulting biotinylated concatemer can then be immobilized on a biotinylated surface through avidin or streptavidin. As another example, the concatemer can be immobilized through hybridization to one or more oligonucleotides bound to the surface. As yet another example, the concatemer can be immobilized through electrostatic interactions with a positively charged surface. As yet another example, the concatemer can be covalently attached to the surface, e.g., through one or more functional groups included in the rolling circle amplification primer and/or in the concatemer itself; for example, a click chemistry group can be included in one or more nucleotides in the primer and/or in one or more nucleotides incorporated into the concatemer during rolling circle amplification. See, e.g., U.S. patent application Ser. No. 17/575,094, which describes exemplary suitable surfaces and attachment of concatemers thereto and which is hereby incorporated by reference herein in its entirety for all purposes. Where a population of concatemers is immobilized on the surface, their positions can be preselected such that they are a sufficient distance apart such that signals generated for a given concatemer during a sequencing process are resolvable from signals generated by other concatemers. Similarly, where a population of concatemers is immobilized at randomly determined positions on the surface, their density can be controlled such that at least some of the concatemers are a sufficient distance apart such that signals generated for a given concatemer during a sequencing process are resolvable from signals generated by other concatemers (e.g., by diluting the concatemers prior to deposition and/or by limiting the density of available attachment sites on the surface).

The forward and reverse strands within the concatemer will, under certain conditions, hybridize to each other as they are produced, such that the concatemer includes double-stranded regions separated by single-stranded regions that are complementary to the single-stranded loops in the rolling circle amplification template. Employing a strand-displacing polymerase in the first and second sequencing processes can thus be advantageous. (This polymerase can be the same as or different than any strand-displacing polymerase employed in a preceding rolling circle amplification reaction.) Techniques to reduce secondary structure formation by discouraging or eliminating hybridization of the forward and reverse strands in the concatemer can also be employed, instead of or in conjunction with use of a strand displacing polymerase. For example, a single-stranded DNA binding protein (e.g., E. coli single-stranded DNA binding protein) can be provided in quantities sufficient to maintain the forward and reverse strands in single-stranded form, e.g., during the rolling circle amplification and/or sequencing processes. Various suitable single-stranded binding proteins (SSBs) are known in the art, as are techniques for their purification. In addition, single-stranded DNA binding proteins, e.g., from E. coli, are commercially available from suppliers such as Thermo Fisher Scientific and AS One International.

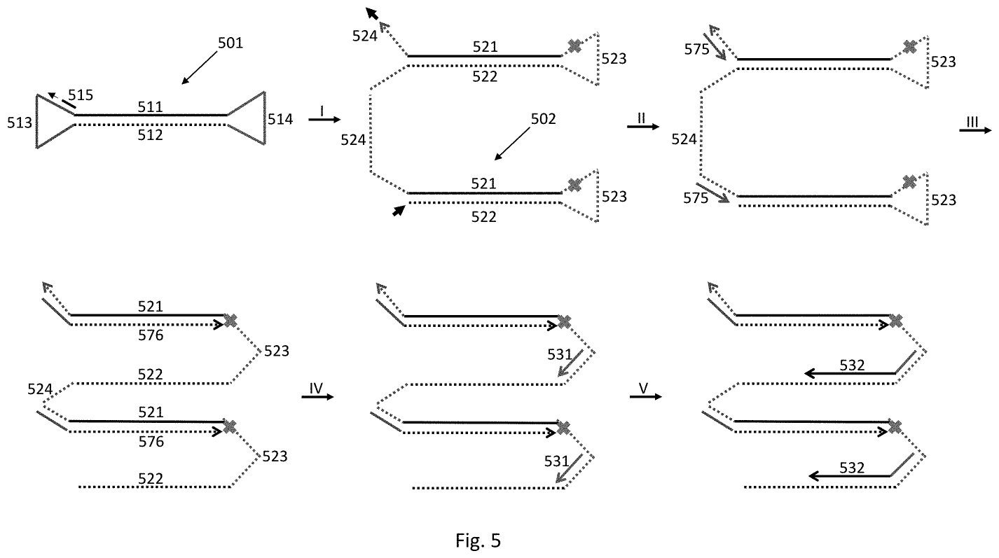

As another example, a masking strand complementary to one of the target strands can be produced to ensure that the other strand is in single-stranded form and is thus readily accessible for sequencing with a polymerase that lacks or has weak strand displacement activity. FIG. 5 schematically illustrates an embodiment of a method of the invention in which a masking strand is employed to facilitate sequencing with a polymerase that lacks strong strand displacement activity. An asymmetric circular rolling circle amplification template 501 is provided. Circular template 501 includes a central target region including self-complementary forward (511) and reverse (512) regions. The template is capped on one end with first connecting region 513 and capped on the other end with second connecting region 514. First connecting region 513 has a different nucleic acid sequence than second connecting region 514. The connecting regions can have sequences with various functions, such as barcodes, priming sites, restriction sites, recognition sites, etc. Note that that while the connectors or connecting regions are shown as corresponding to the single-stranded loop regions of template 501, in many cases, the connectors will also have self-complementary regions that extend into the double-stranded portion of circular nucleic acid 501, e.g., where hairpin adapters each having a single-stranded loop and double-stranded stem are ligated to a double-stranded fragment to form the circular nucleic acid. (See, e.g., the example illustrated in FIG. 4.) One or more priming sites, barcodes, and other useful sequences (or the complement thereof) can be included in the single-stranded or double-stranded portion (or both) of such adapters and therefore in the resulting connecting regions. One of the two connectors (513, in this example) has a rolling circle amplification initiation site such as a rolling circle amplification primer binding site, to which primer 515 is hybridized. The amplification primer binding site is typically, but not necessarily, located in a single-stranded loop region of the connecting region.

Here, in step I, rolling circle amplification primer 515 is extended with a strand-displacing polymerase such as phi29. The polymerase proceeds repeatedly around circular template 501 in the direction indicated by the small arrow, forming concatemer 502 with the repeating structure: first adapter region 523-forward strand 521-second adapter region 524-reverse strand 522. Only a small portion from the middle of the concatemer is shown in the figure; the arrows at both ends of concatemer 502 indicate that there are typically many copies of the repeating unit, for example, hundreds to thousands. The number of copies can be any suitable number, for example tens, hundreds, thousands, or tens of thousands of copies. Here, first adapter region 523 is the complement of first connecting region 513, forward strand 521 is the complement of reverse strand 512 in rolling circle template 501 and is thus substantially identical in sequence to forward strand 511 of 501, second adapter region 524 is the complement of second connecting region 514, and reverse strand 522 is the complement of forward strand 511 in rolling circle template 501 and is thus substantially identical in sequence to reverse strand 512 of 501. While the target segments are shown as directly connected to segments complementary to the single-stranded loop regions of the circular nucleic acid, it is understood that there can be, and typically will be, other intervening sequences between these segments. For example, where stem-loop adapters are used to construct template 501, the complement of one strand of the stem of the adapter would be present between the complement of the loop and the forward target region in the resulting concatemer. As described above for the rolling circle template, these intervening sequences can be used structurally (e.g. self-complementary regions), or the intervening sequences can have specific functions, e.g. cut sites, primer binding sites, recognition sites, or barcodes including molecular barcodes or unique molecular identifiers (UMIs).

This rolling circle amplification (RCA) process can be carried out in solution or can be carried out by extending an RCA primer that is bound to a substrate. Where the RCA primer is bound to a substrate, the result is a concatemer that is bound to the substrate through the primer. The primer can be bound covalently or non-covalently to the surface, e.g., as noted herein or using techniques well known in the art. If the RCA process is carried out in solution, the concatemers produced can be deposited and immobilized onto a substrate, e.g., as noted herein or using techniques well known in the art.

In step II, masking primer 575 hybridizes to a binding site in second adapter region 524. This primer binding site on second adapter region 524 is the complement of a sequence that is included in second connecting region 514 but not in first connecting region 513. In step III, masking primer 575 is extended, typically by a strand-displacing polymerase, to form first masking strand 576 that is complementary to forward strand 521. Extension of masking primer 575 is halted before it proceeds through the entirety of first adapter region 523, such that resulting first masking strand 576 is not complementary to the entirety of first adapter region 523. In the scheme illustrated in FIG. 5, extension is stopped at the site indicated by an X in first adapter region 523. Extension can be blocked using any of a variety of approaches, which can be combined if desired. Exemplary approaches are detailed below. In addition, for any of these exemplary approaches, optionally extension can be carried out at low temperature and/or a polymerase with relatively weak strand-displacement activity can be employed to facilitate halting extension at the desired point.

In one exemplary approach, extension can be halted by hybridizing an oligonucleotide that blocks strand displacement to the single-stranded loop region of the first adapter regions. Exemplary oligonucleotides that can block strand displacement by hybridizing strongly to their binding site include, e.g., oligomers comprising locked nucleic acid (LNA) and/or peptide nucleic acid (PNA) residues. Other exemplary blocking oligonucleotides can form one or more inter-strand cross-links with the first adapter region. For example, a blocking oligonucleotide can include at least one 5-bromo-deoxyuridine (available, e.g., from Integrated DNA Technologies, Inc.) that forms a cross-link with the first adapter region upon exposure to ultraviolet light.

As another example, extension can be halted by including at least one non-natural nucleotide in the first adapter region and extending the masking primer under conditions that exclude the complement of the at least one non-natural nucleotide. A variety of non-natural bases that do not effectively base pair with natural bases are known in the art and can be used in the practice of the present invention. For example, one or more nucleotide residues that include isocytosine (isoC) can be included in the loop region of one of the stem-loop adapters used to make the rolling circle amplification template, such that the first connecting region of the resulting rolling circle amplification template includes one or more isoC. The rolling circle amplification primer is extended in a mixture that includes a nucleotide comprising isoguanine (isoG), such that the resulting concatemer includes one or more isoG. When the masking primer is extended, no isoC is provided in the reaction mixture, so extension cannot proceed past the template position(s) occupied by isoG.

As yet another example, a nick can be introduced into the first adapter region before the masking primer is extended, to halt extension at the nick. Nicking can be performed after or, more typically, before hybridization of the masking primer to the concatemer. The nick can be introduced, e.g., using an endonuclease with a specific recognition site. A variety of suitable site-specific endonucleases are known in the art and are commercially available, e.g., from New England Biosciences, Inc. As one example, a recognition site for a nicking endonuclease can be designed into the double-stranded stem portion of the first adapter region, or an oligonucleotide can be hybridized to the single-stranded loop portion of the first adapter region to create a recognition site for the nicking endonuclease. Either altered restriction enzymes that hydrolyze only one strand of the duplex or naturally occurring nicking endonucleases can be employed. As another example, an oligonucleotide can be hybridized to the single-stranded loop portion of the first adapter region to create a recognition site for an endonuclease that cleaves both strands (cleaving the oligonucleotide and leaving a nick in the adapter region). For example, a methylated oligonucleotide complementary to a single-stranded portion of the first adapter region can create a site for cleavage by FspEI (New England Biosciences, Inc.). As yet another example, Tth Argonaute (New England Biosciences, Inc.) can be employed with a 5′-phosphorylated single-stranded DNA guide to introduce a nick in the portion of the adapter region that has a strand complementary to the guide.

In some embodiments, e.g., where sequencing is performed with a SBB technique where signal from a fluorescently labeled cognate nucleotide binding to the next available base in the template is detected to identify the next correct nucleotide in the sequence (and particularly where multiple targets are being sequenced simultaneously at different positions on the surface of a flow cell or other substrate), keeping the fragments resulting from nicking the concatemer in proximity to each other can increase the intensity of signal observable from that cluster of fragments (i.e., for that particular target nucleic acid). The fragments produced by nicking the concatemer are optionally held together by hybridizing one or more staple oligonucleotides similar to those used in DNA origami to the fragments. Such hybridization can bridge different fragments, keeping the fragments resulting from a given concatemer tightly clustered.

Each staple oligonucleotide is capable of binding (directly or indirectly) simultaneously to two or more fragments to maintain them in proximity to each other. In various exemplary designs, a staple oligonucleotide can be hybridized directly to adapter regions of two or more fragments, can be hybridized to a fragment and to another staple oligonucleotide, and/or can be hybridized to a fragment and bound to an intermediate. For example, one portion of a staple oligonucleotide (e.g., one end of the staple oligonucleotide) can hybridize to a single-stranded portion of the first adapter region while another portion of the staple oligonucleotide (e.g., its other end) can hybridize to a single-stranded portion of the second adapter region, e.g., on a different fragment. As another example, one portion of a staple oligonucleotide (e.g., one end of the staple oligonucleotide) can hybridize to a single-stranded portion of the first adapter region while another portion of the staple oligonucleotide (e.g., its other end) can hybridize to a single-stranded portion of another instance of the first adapter region; the portions of the first adapter regions to which the staple oligonucleotide hybridizes optionally have the same sequence. Similarly, one staple oligonucleotide can hybridize to single-stranded portions of two different instances of the second adapter region. In certain aspects, portions of staple oligonucleotides may hybridize to each other or to an intermediate. For example, portions of two or more staple oligonucleotides may each hybridize to the same single intermediate oligonucleotide or to different intermediate oligonucleotides, e.g., that are presented by a particle. Staple oligonucleotides can be functionalized (e.g., at their 3′ and/or 5′ ends and/or internally) for cross-linking to each other or thorough an intermediate. For example, biotinylated staple oligonucleotides can be cross-linked through streptavidin (e.g., through addition of streptavidin after hybridizing staple oligonucleotides to first and/or second adapter regions). In another example, staple oligonucleotides can be functionalized with a click chemistry group, such as a strain-promoted click chemistry group, and can be cross-linked through a multivalent intermediate presenting multiple instances of the complementary click chemistry partner. Exemplary strain-promoted click chemistry groups and partners include, but are not limited to, dibenzocyclooctyne (DBCO) and azide, trans-cyclooctene (TCO) and tetrazine, and derivatives thereof. For example, DBCO-functionalized staple oligonucleotides can be cross-linked by a dendrimer presenting a plurality of azides.

A single oligonucleotide can optionally serve as both a primer and a staple oligonucleotide. For example, the 5′ end of the first sequencing primer can hybridize to the second adapter region while the 3′ end of the first sequencing primer hybridizes to the first adapter region (such that the first sequencing primer also serves as a staple oligonucleotide), and/or the 5′ end of the masking primer can hybridize to the first adapter region while the 3′ end of the masking primer hybridizes to the second adapter region (such that masking primer also serves as a staple oligonucleotide). It will be evident that the portion of the adapter regions complementary to the sequencing, masking, and staple portions of such primers will generally not overlap each other so that the primers are not competing for their binding sites.

In the example illustrated in FIG. 5, after synthesis of first masking strand 576, in step IV first sequencing primer 531 is hybridized to a primer binding site in first adapter region 523. This primer binding site on first adapter region 523 is the complement of a sequence that is included in first connecting region 513 but that is not present in second connecting region 514. The sequencing process can be carried out on a concatemer or a plurality of concatemers (e.g., thousands to millions to billions) immobilized on a substrate. The sequencing process can be by a stepwise sequencing process such as SBS, SBB, or sequencing by ligation. In the exemplary embodiment illustrated in FIG. 5, first sequencing primer 531 is extended in step V to form nascent strand 532, with extension proceeding in the direction indicated by the arrowhead. The sequencing process proceeds from sequencing primer 531 into reverse strand 522 of the target sequence until the first sequencing process is halted. This process produces the first read.

Where paired-end reads from the target nucleic acid sequence are desired, any of various approaches can be employed to obtain the second read. Two exemplary approaches are detailed below.

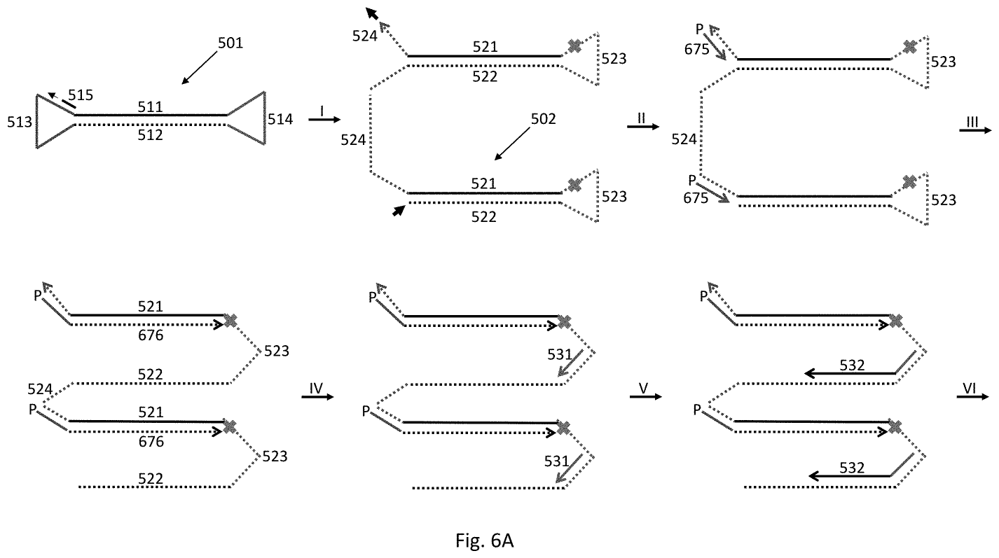

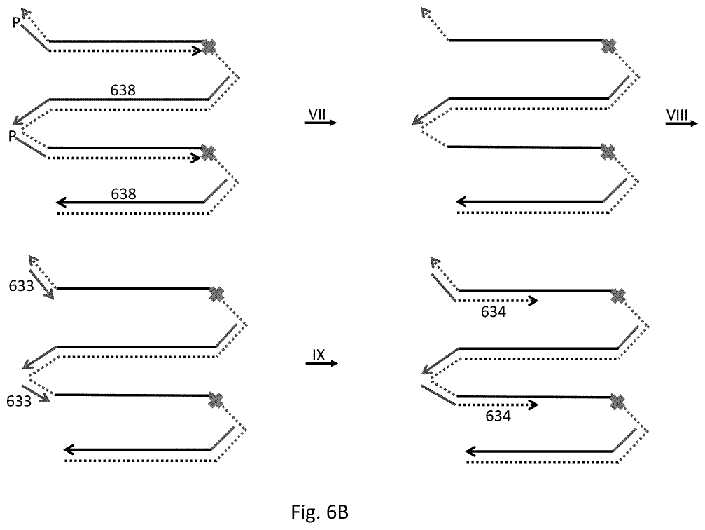

FIGS. 6A-6B schematically illustrate an embodiment in which the second read is obtained from the other strand of the target within the concatemer. In this embodiment, masking primer 675 comprises a 5′ phosphate group, and thus first masking strand 676 does as well. In other regards, steps I-V are performed as detailed above for FIG. 5. After sequencing from first sequencing primer 531 is complete, nascent strand 532 produced by sequencing from first sequencing primer 531 is extended in step VI to produce second masking strand 638. Extension of nascent strand 532 is halted before it proceeds through the entirety of second adapter region 524, such that resulting second masking strand 638 is not complementary to the entirety of second adapter region 524. First sequencing primer 531 can be extended using a polymerase that lacks strand displacement activity such that extension stops at the 5′ end of first masking strand 676, or masking primer 675 and thus first masking strand 676 can include LNA or PNA residues (or other modifications that result in tight binding to the concatemer), inter-strand cross-links between masking primer 675 and second adapter region 524 can be formed, or second adapter region 524 can include at least one non-natural nucleotide whose complement is not included in the extension reaction, in order to halt extension as detailed above. After synthesis of second masking strand 638, first masking strand 676 is removed in step VII, e.g., by digestion with lambda exonuclease or a similar enzyme that catalyzes 5′ to 3′ nucleotide removal from double-stranded DNA having a 5′ phosphate.

In step VIII, second sequencing primer 633 hybridizes to a primer binding site in second adapter region 524. This primer binding site on second adapter region 524 is the complement of a sequence that is included in second connector 514 and that is not present in first connector 513. The second sequencing process is carried out from second sequencing primer 633 in step IX. The second sequencing process is typically carried out in the same manner as the first sequencing process, but could be carried out with a different process. In the exemplary embodiment illustrated in FIGS. 6A-6B, second sequencing primer 633 is extended to form nascent strand 634, with extension proceeding in the direction indicated by the arrowhead. The second sequencing process is carried out until the process is halted. It produces the second read. The second read is from a strand that is complementary to the strand from which the first read is obtained. The second read extends in from the opposite end of the target nucleic acid of interest than the first read. The combination of the first read and the second read provides a paired-end sequence of the target sequence of interest.

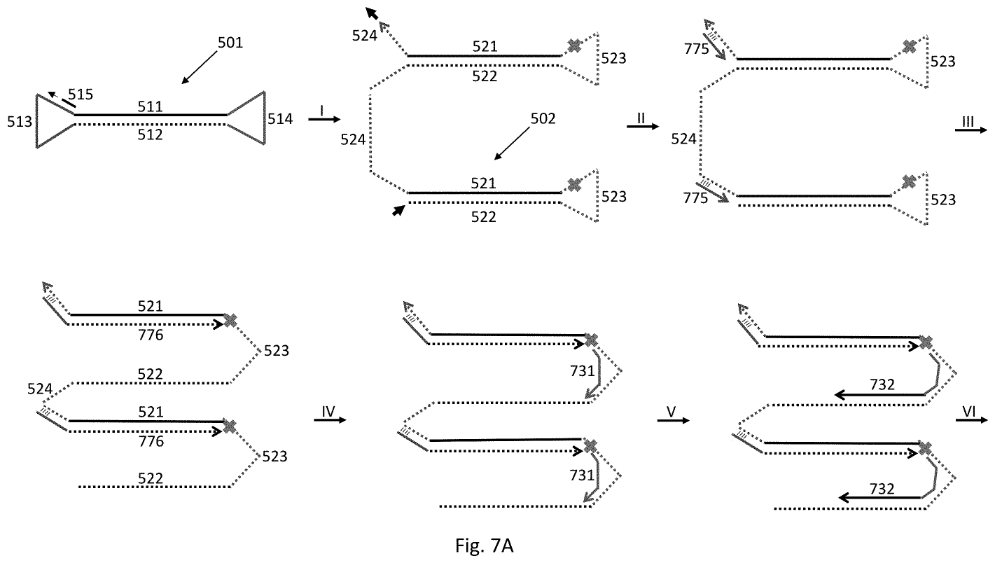

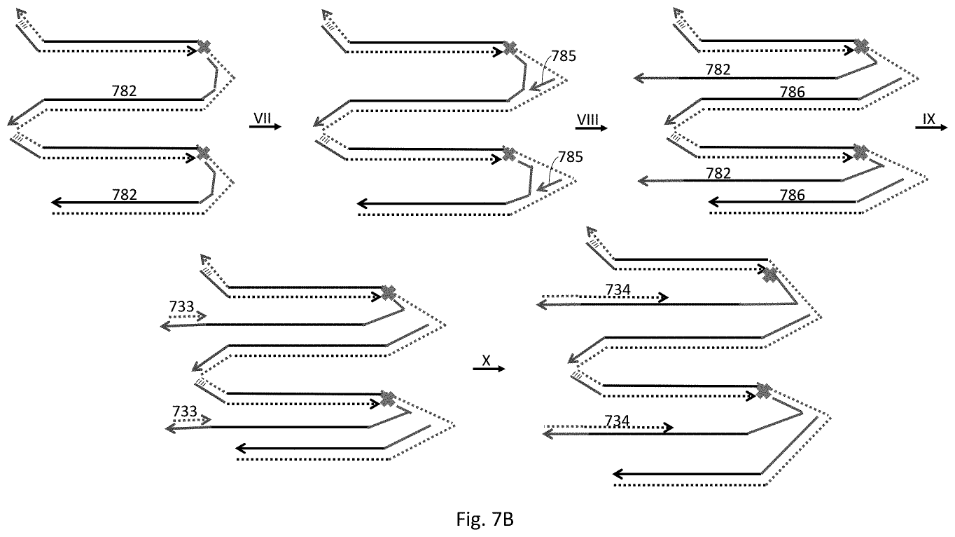

FIGS. 7A-7B schematically illustrate an embodiment in which the second read is produced from a strand formed by extending the first sequencing primer rather than directly from the concatemer. In this embodiment, masking primer 775, and thus also first masking strand 776, include LNA residues, PNA residues, or other modifications that result in strong binding to second adapter region 524. In other regards, steps I-III are performed as detailed above for FIG. 5.

In the example illustrated in FIGS. 7A-7B, after synthesis of first masking strand 776, in step IV first sequencing primer 731 is hybridized to first adapter region 523. First sequencing primer 731 comprises a region at or near its 5′ end and a region at or near its 3′ end that each hybridize to first adapter region 523. These 5′ and 3′ regions of first sequencing primer 731 flank a central region that is not complementary to and does not hybridize to first adapter region 523. The primer binding subsites on first adapter region 523 are separated by a region that is not complementary to first sequencing primer 731, and thus a portion of first adapter region 523 remains single-stranded. In the exemplary embodiment illustrated in FIGS. 7A-7B, first sequencing primer 731 is extended in step V to form nascent strand 732, with extension proceeding in the direction indicated by the arrowhead. The sequencing process can be a stepwise sequencing process such as SBS, SBB, or sequencing by ligation, and can be carried out on a concatemer or a plurality of concatemers immobilized on a substrate. The sequencing process proceeds from first sequencing primer 731 into reverse strand 522 of the target sequence until the first sequencing process is halted. This process produces the first read.

As illustrated in FIGS. 7A-7B, in step VI nascent strand 732 produced by sequencing from first sequencing primer 731 is further extended to produce extended strand 782 that is complementary to reverse strand 522. Extension stops when the tight binding 5′ end of first masking strand 776 is reached, such that extended strand 782 is not complementary to the entirety of second adapter region 524 (although it is typically complementary to a portion of it, to provide a convenient universal binding site for second sequencing primer 733). It will be evident that the other techniques detailed above for halting extension can be employed in addition to or instead of inclusion of LNA in first masking primer 775 and first masking strand 776 (e.g., nicking of second adapter region 524 or inclusion of a non-natural nucleotide in second adapter region 524 and exclusion of its complementary nucleotide from the extension reaction).

In step VII, displacement primer 785 is hybridized to the single-stranded portion of first adapter region 523 that is between the regions that bind first sequencing primer 731. In step VIII, second masking strand 786 is produced as displacement primer 785 is extended, typically with a strand-displacing polymerase, to displace extended strand 782 from reverse strand 522. The 5′ region of extended strand 782 remains hybridized to first adapter region 523. In the embodiment illustrated in FIGS. 7A-7B, extension of displacement primer 785 stops when the tight binding 5′ end of first masking strand 776 is reached. Again, it will be evident that the other techniques detailed above for halting extension can be employed in addition to or instead of inclusion of LNA in first masking primer 775 and first masking strand 776 (e.g., nicking of second adapter region 524 or inclusion of a non-natural nucleotide in second adapter region 524 and exclusion of its complementary nucleotide from the extension reaction). In some embodiments, extension is allowed to proceed through second adapter region 524; in such embodiments, extension can instead be stopped in first adapter region 523 using the techniques detailed above (e.g., nicking of first adapter region 523 or inclusion of a non-natural nucleotide in first adapter region 523 and exclusion of its complementary nucleotide from the extension reaction).

In step IX, second sequencing primer 733 hybridizes to a primer binding site in displaced extended strand 782. The second sequencing process is carried out from second sequencing primer 733 in step X. The second sequencing process is typically carried out in the same manner as the first sequencing process, but could be carried out with a different process. In the exemplary embodiment illustrated in FIGS. 7A-7B, second sequencing primer 733 is extended to form nascent strand 734, with extension proceeding in the direction indicated by the arrowhead. The second sequencing process is carried out until the process is halted. It produces the second read. The second read is from a strand that is complementary to the strand from which the first read is obtained. The second read extends in from the opposite end of the target nucleic acid of interest than the first read. The combination of the first read and the second read provides a paired-end sequence of the target sequence of interest.

While the preceding examples have been described in terms of paired-end sequencing, it will be evident that each of these methods can be employed for determining the nucleic acid sequence of a single region of the target simply by performing only those steps that result in sequencing from the first sequencing primer to obtain the first read, and not continuing on with steps that result in sequencing from the second sequencing primer to obtain the second read. Such methods are also features of the invention.

### Generating Rolling Circle Amplification Templates

In general, nucleic acid rolling circle amplification templates of particular use in the context of the present invention include a double-stranded region (e.g., a double-stranded nucleic acid insert containing a target nucleic acid sequence of interest) with a hairpin at each end. Such constructs can be generated using any suitable method. In some embodiments, a hairpin end is attached to a double-stranded nucleic acid fragment by ligating a hairpin adapter to a compatible end of the fragment, e.g., via blunt-end or sticky-end ligation, as is known in the art. The ligation of a hairpin adapter creates a nucleic acid construct comprising a single-stranded region that connects the two strands of the double-stranded nucleic acid insert (connecting the 3′ end of a first strand of the insert with the 5′ end of the hybridized complementary strand of the insert, e.g., through the stem of the hairpin adapter, connecting the 3′ end of the adapter with the 5′ end of one strand of the insert and the 5′ end of the adapter with the 3′ end of the other strand of the insert, as illustrated in FIG. 4). The single-stranded regions can be used for priming and/or capture, as desired. Other methods for generating a hairpin end on a nucleic acid may be employed.

Circular nucleic acids that find use in the present disclosure include SMRTBELL® templates, which are nucleic acids having a central double-stranded region, and having hairpin regions at each end of the double-stranded region. The preparation and use of cyclic templates such as SMRTBELL® templates are described, for example, in U.S. Pat. Nos. 8,153,375, 8,236,499, and Travers et al. (2010) Nucl. Acids Res. 38(15):e159, the full disclosures of which are hereby incorporated herein by reference for all purposes. One advantage of the SMRTBELL® template is that it can be made from a library of double-stranded nucleic acid, e.g. DNA, fragments. For example, a sample of genomic DNA can be fragmented into a library of DNA fragments, by known methods such as by shearing or by use of restriction enzymes. The library of DNA fragments can be ligated to hairpin or other stem-loop adapters at each end of the fragment to produce a library of SMRTBELL® circular templates. The hairpin adapters provide single-stranded regions within the hairpins, which provide useful locations for rolling circle amplification primer binding sites and for the complement of sequencing primer binding sites. By using the same pair of hairpin adapters for all of the fragments, the hairpin adapters provide a position for universal priming of all of the target sequences.

As described above with regard to FIG. 3, ligating a mixture of two different hairpin adapters to double-stranded target fragments results in a mixture of symmetric circular nucleic acids (having the same adapter at both ends) and asymmetric circular nucleic acids (having different adapters at the two ends). While this mixture can be used for sequencing of the target(s) as detailed above, the asymmetric circular constructs can be purified from the mixture, e.g., as detailed in U.S. Pat. No. 10,920,268, which is hereby incorporated by reference herein in its entirety for all purposes. For example, symmetric circular nucleic acids including two copies of a first hairpin adapter and asymmetric circular nucleic acids including the first hairpin adapter and a second hairpin adapter can be captured on beads bearing a capture primer complementary to the first adapter. Symmetric circular nucleic acids that have two copies of the second adapter are not captured and are thus removed from the mixture. A primer complementary to the second adapter can then be extended with a strand-displacing polymerase, and this extension of the primer elutes the asymmetric nucleic acids from the beads. Other techniques for purifying asymmetric circular nucleic acids are also detailed in U.S. Pat. No. 10,920,268.

It will be evident that in addition to or instead of purifying an asymmetric nucleic acid from a mixture of symmetric and asymmetric constructs, desired concatemers can be purified from a mixture of concatemers. For example, a mixture of a first hairpin adapter comprising a rolling circle amplification primer binding site and a second hairpin adapter lacking the rolling circle amplification primer binding site can be ligated to double-stranded target fragments, resulting in a mixture of symmetric circular nucleic acids (having the same adapter at both ends) and asymmetric circular nucleic acids (having different adapters at the two ends). When rolling circle amplification is performed, only asymmetric constructs and those symmetric constructs having two first adapters will be amplified; symmetric constructs having two second adapters cannot hybridize to the primer and thus will not be amplified. The resulting concatemers can be exposed to a solid support (e.g., beads, the surface of a flow cell or other sequencing substrate, or the like) through binding of an oligonucleotide immobilized on the solid support to a binding site in the region of the concatemer that is complementary to the second adapter. Only concatemers resulting from amplification of the asymmetric constructs will thus be captured. These concatemers can be used as sequencing templates as detailed herein, or they can be subjected to multiple displacement amplification and the resulting concatemeric products can be used as sequencing templates as detailed herein.

Rather than purifying asymmetric nucleic acids from a mixture of symmetric and asymmetric constructs, techniques for constructing a population of asymmetric nucleic acids can be employed. Exemplary suitable methods for generating asymmetric circular nucleic acids are described in, e.g., U.S. Pat. No. 10,370,701, which is hereby incorporated by reference herein in its entirety for all purposes. For example, a symmetric circular nucleic acid comprising a nick between the hairpin adapter and the double-stranded insert at each end is constructed. Extension is performed from the nick, and a different hairpin adapter is ligated to the free end of the resulting product. Other methods for producing nucleic acid constructs suitable for use as the asymmetric rolling circle amplification templates of the invention are provided, for example, in U.S. patent application publication no. 2012/0196279, which is hereby incorporated by reference herein in its entirety for all purposes.

Some useful methods for generating rolling circle amplification templates for use in the methods of the invention begin with double-stranded nucleic acid fragments having defined ends, which could be blunt ends or ends with known overhang sequences (5′ or 3′ overhangs). These nucleic acid fragments can be of any size or size range and can include DNA, RNA, DNA-RNA hybrids (e.g., molecules produced by first-strand synthesis during preparation of cDNA that have one mRNA strand and one complementary DNA strand), genomic DNA, cDNA, mRNA, tRNA, etc. In some embodiments, the nucleotide sequence of the fragments is not known.

In certain embodiments, the double-stranded nucleic acid fragments used in methods and compositions of the present disclosure comprise nucleic acids obtained from a sample. The sample may comprise any number of things, including, but not limited to: bodily fluids (including, but not limited to, blood, urine, serum, lymph, saliva, anal and vaginal secretions, perspiration and semen) and cells of virtually any organism (e.g., mammalian species including humans); environmental samples (including, but not limited to, air, agricultural, water and soil samples); biological warfare agent samples; research samples; the products of an amplification reaction (including both target and signal amplification, such as PCR amplification reactions); purified samples (e.g., such as purified genomic DNA, raw samples (bacteria, virus, genomic DNA, etc.)). As will be appreciated by those in the art, virtually any experimental manipulation may have been done on the samples.

Genomic DNA, when used in the disclosed methods, can be prepared from any source basically by three steps: cell lysis, deproteinization and recovery of DNA. These steps are adapted to the demands of the application, the requested yield, purity, and molecular weight of the DNA, and the amount and history of the source. Further details regarding the isolation of genomic DNA can be found in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2008 (“Sambrook”); Current Protocols in Molecular Biology, F. M. Ausubel et al., eds., Current Protocols, a joint venture between Greene Publishing Associates, Inc. and John Wiley & Sons, Inc (supplemented through 2021) (“Ausubel”); Kaufman et al. (2003) Handbook of Molecular and Cellular Methods in Biology and Medicine Second Edition Ceske (ed) CRC Press (Kaufman); and The Nucleic Acid Protocols Handbook Ralph Rapley (ed) (2000) Cold Spring Harbor, Humana Press Inc (Rapley). In addition, many kits are commercially available for the purification of genomic DNA from cells, including Wizard™ Genomic DNA Purification Kit, available from Promega; Aqua Pure™ Genomic DNA Isolation Kit, available from BioRad; Easy-DNA™ Kit, available from Thermo Fisher Scientific; and DnEasy™ Tissue Kit, which is available from Qiagen. Alternatively, or additionally, target nucleic acid segments may be obtained through targeted capture protocols, e.g., where target nucleic acids are obtained initially as single-stranded segments on microarrays or other capture techniques, followed by amplification of the captured material to generate double-stranded sample materials. A variety of such capture protocols have been described in, e.g., Hodges E, et al. Nat. Genet. 2007 Nov. 4, Olson M., Nature Methods 2007 November; 4(11):891-2, Albert T J, et al. Nature Methods 2007 November; 4(11):903-5, and Okou D T, et al. Nature Methods 2007 November; 4(11):907-9.

Nucleic acids that can be used in the methods described herein can also be derived from a cDNA, e.g. cDNAs prepared from mRNA obtained from, e.g., a eukaryotic subject or a specific tissue derived from a eukaryotic subject. Data obtained from sequencing the nucleic acid targets derived from a cDNA library, e.g., using a high-throughput sequencing system, can be useful in identifying, e.g., novel splice variants of a gene of interest or in comparing the differential expression of, e.g., splice isoforms of a gene of interest, e.g., between different tissue types, between different treatments to the same tissue type or between different developmental stages of the same tissue type.

mRNA can typically be isolated from almost any source using protocols and methods described in, e.g., Sambrook and Ausubel. The yield and quality of the isolated mRNA can depend on, e.g., how a tissue is stored prior to RNA extraction, the means by which the tissue is disrupted during RNA extraction, or on the type of tissue from which the RNA is extracted. RNA isolation protocols can be optimized accordingly. Many mRNA isolation kits are commercially available, e.g., from Thermo Fisher Scientific and BioChain. In addition, mRNA from various sources, e.g., bovine, mouse, and human, and tissues, e.g. brain, blood, and heart, is commercially available from, e.g., BioChain (Hayward, Calif.) and Takara Bio (San Jose, Calif.).

Once the purified mRNA is recovered, reverse transcriptase is used to generate cDNAs from the mRNA templates. Methods and protocols for the production of cDNA from mRNAs, e.g., harvested from prokaryotes as well as eukaryotes, are elaborated in cDNA Library Protocols, I. G. Cowell, et al., eds., Humana Press, New Jersey, 1997, Sambrook and Ausubel. In addition, many kits are commercially available for the preparation of cDNA, including the Cells-to-cDNA™ II, RETROscript™, and CloneMiner™ cDNA Library Construction Kits (Thermo Fisher Scientific) and the Universal RiboClone® cDNA Synthesis System (Promega). Many companies, e.g., Creative Biogene and GenScript, offer cDNA synthesis services.

In some embodiments of the invention described herein, nucleic acid fragments are generated from a genomic DNA or a cDNA. There exist a plethora of ways of generating nucleic acid fragments from a genomic DNA, a cDNA, or a DNA concatemer. These include, but are not limited to, mechanical methods, such as sonication, mechanical shearing, nebulization, hydroshearing, and the like; chemical methods, such as treatment with hydroxyl radicals, Cu(II):thiol combinations, diazonium salts, and the like; enzymatic methods, such as exonuclease digestion, restriction endonuclease digestion, transposon cleavage and tagging, and the like; and electrochemical cleavage. These methods are further explicated, e.g., in Sambrook and Ausubel.

In some embodiments, nucleic acid molecules are obtained from a sample and fragmented for use in methods of the present disclosure. The fragments may further be modified in accordance with any methods known in the art and described herein. Nucleic acid fragments may be generated by fragmenting source nucleic acids, such as genomic DNA, using any method known in the art. In one embodiment, shear forces during lysis and extraction of genomic DNA generate fragments in a desired range. Also encompassed by the invention are methods of fragmentation utilizing restriction endonucleases.

Double-stranded nucleic acid fragments can be any length that is desired for subsequent uses, e.g., cloning, transformation, enrichment, sequencing, etc. In certain embodiments, the fragments can be from about 10 to about 50,000 base pairs (bp) in length and any range therebetween, e.g., from about 100 to about 40,000 bp, from about 300 to 30,000 bp, from about 500 to 20,000 bp, from about 800 to 10,000 bp, from about 1,000 to 8,000 bp, etc. In certain embodiments, the average size of the double-stranded nucleic acid fragments is at least about 100 bp in length, at least about 200, at least about 300, at least about 500, at least about 1,000, at least about 1,500, at least about 2,000, at least about 5,000, at least about 10,000, at least about 20,000, etc. In some embodiments, the average length of the target nucleic acid sequence that is inserted into the rolling circle amplification template is between 50 and 20,000 bp, e.g., between 50 and 10,000 bp, between 50 and 5,000 bp, 50 and 2,000 bp, between 50 and 1,000 bp, between 50 and 500 bp, between 50 and 300 bp, between 50 and 200 bp, between 200 and 800 bp, or between 200 and 500 bp. The average length of the forward and reverse target nucleic acid sequence in the resulting concatemer in such embodiments is thus each between 50 and 20,000 nucleotides, e.g., between 50 and 10,000 nucleotides, between 50 and 5,000 nucleotides, 50 and 2,000 nucleotides, between 50 and 1,000 nucleotides, between 50 and 500 nucleotides, between 50 and 300 nucleotides, between 50 and 200 nucleotides, between 200 and 800 nucleotides, or between 200 and 500 nucleotides.

In certain embodiments, the fragments are treated to produce blunt ends that are compatible with ligation to adapters having a compatible blunt end. Any suitable method for producing blunt ends may be employed, including treatment with one or more enzyme having 5′ and/or 3′ single strand exonuclease activity (e.g., E. coli Exonuclease III) and/or performing a fill-in reaction to extend 3′ recessed ends (e.g., with T4 DNA polymerase). No limitation in this regard is intended. In other embodiments, the fragments have sticky ends, e.g., left by treatment with a restriction endonuclease or other nuclease, or a 3′ A added by Taq polymerase, and the adapters have overhangs complementary to these ends. It will be evident that the two ends of the fragments can have different sticky ends or that one can be blunt while the other is sticky, and that one adapter will be compatible with one end and the other adapter with the other.

### Rolling Circle Amplification

Rolling circle amplification methods that can be employed to produce concatemers from asymmetric circular nucleic acids for use as sequencing templates in the invention are well known in the art. See, for example, U.S. Pat. No. 5,648,245 “Concatemer library by RCA” by Fire et al., U.S. patent application publication no. 20050069939 “Amplification of polynucleotides by rolling circle amplification” by Wang et al., and U.S. Pat. No. 9,290,800 “Targeted rolling circle amplification” by Turner et al., which are incorporated by reference herein in their entirety for all purposes. Rolling circle amplification from oligonucleotides attached to a substrate are described, for example, in U.S. Pat. No. 6,274,320 by Rothberg and Bader and U.S. patent application publication no. 20060024711 by Lapidus, which are incorporated by reference herein in their entirety for all purposes.

### Nucleic Acid Sequencing Processes

Suitable sequencing processes for use in the provided methods include, but are not limited to, sequencing by binding, sequencing by synthesis (sequencing by incorporation), pH-based sequencing, sequencing by polymerase monitoring, sequencing by hybridization, and other methods of massively parallel sequencing or next-generation sequencing. Suitable surfaces for carrying out sequencing include, but are not limited to, a planar substrate, a hydrogel, a nanohole array, a microparticle, a nanoparticle, or a surface within a flow cell. Exemplary sequencing platforms including methods, reagents and solid-phase surfaces are set forth below and in the cited references.

In one aspect, sequencing is performed with a sequencing by binding (SBB) technique. Exemplary particularly useful sequencing by binding reactions are described in U.S. Pat. Nos. 10,077,470, 10,443,098, 10,400,272, and 10,975,427 and U.S. patent application publication nos. 20190119742, 20180187245, and 20200032322, each of which is incorporated by reference herein in its entirety. Generally, methods for determining the sequence of a template nucleic acid molecule through sequencing by binding can be based on formation of a ternary complex (between polymerase, primed nucleic acid, and cognate nucleotide) under specified conditions. The method can include an examination phase followed by a nucleotide incorporation phase.

The examination phase in a sequencing by binding procedure can be carried out in a flow cell having at least one template nucleic acid molecule (e.g., a concatemeric RCA product) primed with a primer; contacting the primed template nucleic acid molecule(s) with a first reaction mixture that includes a polymerase and at least one nucleotide type; observing the interaction of polymerase and a nucleotide with the primed template nucleic acid molecule(s), under conditions where the nucleotide is not covalently added to the primer(s); and identifying a next base in each template nucleic acid using the observed interaction of the polymerase and nucleotide with the primed template nucleic acid molecule(s). The interaction between the primed template, polymerase, and nucleotide can be detected in a variety of schemes. For example, the nucleotides can contain a detectable label. Each nucleotide can have a distinguishable label with respect to other nucleotides. Alternatively, some or all of the different nucleotide types can have the same label and the nucleotide types can be distinguished, e.g., based on separate deliveries of different nucleotide types or combinations thereof to the flow cell. In some embodiments, the polymerase can be labeled. Polymerases that are associated with different nucleotide types can have unique labels that distinguish the type of nucleotide to which they are associated. Alternatively, polymerases can have similar labels and the different nucleotide types can be distinguished based on separate deliveries of different nucleotide types to the flow cell (e.g., delivering the labeled polymerase in combination with one or more unlabeled nucleotides at a time).

During the examination phase, discrimination between correct and incorrect nucleotides can be facilitated by ternary complex stabilization. A variety of conditions and reagents can be useful for ternary complex stabilization, e.g., by preventing incorporation of nucleotide and/or preventing dissociation of the ternary complex. For example, the primer can contain a reversible blocking moiety that prevents covalent attachment of nucleotide, cofactors that are required for extension (such as divalent metal ions) can be absent, inhibitory divalent cations that inhibit polymerase-based primer extension can be present, the polymerase that is present in the examination phase can have a chemical modification and/or mutation that inhibits primer extension, and/or the nucleotides can have chemical modifications that inhibit incorporation, such as 5′ modifications that remove or alter the native triphosphate moiety. Conditions such as salt concentration, pH, and temperature can also contribute to ternary complex stability.

The extension phase can then be carried out by creating conditions in the flow cell where a nucleotide can be added to the primer on each template nucleic acid molecule. In some embodiments, this involves removal of reagents used in the examination phase and replacing them with reagents that facilitate extension. For example, examination reagents can be replaced with a polymerase and nucleotide(s) that are capable of extension. Alternatively, one or more reagents can be added to the examination phase reaction to create extension conditions. For example, catalytic divalent cations can be added to an examination mixture that was deficient in the cations, and/or polymerase inhibitors can be removed or disabled, and/or extension competent nucleotides can be added, and/or a deblocking reagent can be added to render primer(s) extension competent, and/or extension competent polymerase can be added. Optionally, the nucleotide that is enzymatically incorporated into the primer strand of the primed template nucleic acid molecule is different from the nucleotide used in the examination step to identify the next correct nucleotide.

Optionally, the polymerase used in the incorporation step is different from the polymerase used in the examination step. Optionally, the incorporated nucleotide is a reversible terminator nucleotide, where primer extension is limited to a single nucleotide incorporation prior to removal of a reversible terminator moiety. Thus, for embodiments employing reversible terminator nucleotides, a deblocking reagent can be delivered to a flow cell (before or after detection occurs). Washes can be carried out between the various delivery steps.

The above examination and extension phases can be carried out cyclically such that in each cycle a single next correct nucleotide is examined (i.e., the next correct nucleotide being a nucleotide that correctly binds to the nucleotide in a template nucleic acid that is located immediately 5′ of the base in the template that is hybridized to the 3′ end of the hybridized primer) and, subsequently, a single next correct nucleotide is added to the primer. Any number of cycles can be carried out including, for example, at least 1, 2, 5, 10, 20, 25, 30, 40, 50, 75, 100, 150 or more cycles. Alternatively or additionally, the number of cycles can be capped at no more than 150, 100, 75, 50, 40, 30, 25, 20, 10, 5, 2, or 1 cycles. This cyclical sequencing process produces a read of all or a portion of the template nucleic acid's sequence.

A sequencing by synthesis (SBS) technique can also be used. This technique generally involves the enzymatic extension of a primer through the iterative addition of nucleotides against a template strand to which the primer is hybridized. Briefly, SBS can be initiated by contacting target nucleic acids, attached to features in a flow cell, with one or more labeled nucleotides, DNA polymerase, etc. Those features where a primer is extended using the target nucleic acid as template will incorporate a labeled nucleotide that can be detected. Optionally, the labeled nucleotides can further include a reversible termination property that terminates further primer extension once a nucleotide has been added to a primer. For example, a nucleotide analog having a reversible terminator moiety can be added to a primer so that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated. Exemplary SBS procedures, reagents and detection instruments that can be readily adapted for use with an array of concatemers in the methods of the present disclosure are described, for example, in Bentley et al., Nature 456:53-59 (2008), WO 04/018497, WO 91/06678, WO 07/123744, U.S. Pat. Nos. 7,057,026, 7,329,492, 7,211,414, 7,315,019, and 7,405,281, and U.S. Pat. App. Pub. No. 2008/0108082, each of which is incorporated herein by reference. Also useful are SBS methods that are commercially available from Illumina, Inc. (San Diego, Calif.).

Some SBS embodiments include detection of a proton released upon incorporation of a nucleotide into an extension product. For example, sequencing based on detection of released protons can use reagents and an electrical detector that are commercially available from Thermo Fisher (Waltham, Mass.) or described in U.S. patent application publication nos. 2009/0026082, 2009/0127589, 2010/0137143, and 2010/0282617, each of which is incorporated by reference.

Other sequencing procedures can be used, such as pyrosequencing. Pyrosequencing detects the release of inorganic pyrophosphate (PPi) as particular nucleotides are incorporated into a nascent primer hybridized to a template nucleic acid strand. See, e.g., Ronaghi, et al., Analytical Biochemistry 242 (1), 84-9 (1996); Ronaghi, Genome Res. 11 (1), 3-11 (2001); Ronaghi et al. Science 281 (5375), 363 (1998); and U.S. Pat. Nos. 6,210,891, 6,258,568, and 6,274,320, each of which is incorporated herein by reference. In pyrosequencing, released PPi can be detected by being converted to adenosine triphosphate (ATP) by ATP sulfurylase, and the resulting ATP can be detected via luciferase-produced photons. Thus, the sequencing reaction can be monitored via a luminescence detection system.

Sequencing by ligation reactions are also useful, including, for example, those described in Shendure et al. Science 309:1728-1732 (2005) and U.S. Pat. Nos. 5,599,675 and 5,750,341, each of which is incorporated by reference. Some embodiments can include sequencing by hybridization procedures as described, for example, in Bains et al., Journal of Theoretical Biology 135 (3), 303-7 (1988); Drmanac et al., Nature Biotechnology 16, 54-58 (1998); Fodor et al., Science 251 (4995), 767-773 (1995); and WO 1989/10977, each of which is incorporated by reference. In both sequencing by ligation and sequencing by hybridization procedures, primers that are hybridized to nucleic acid templates are subjected to repeated cycles of extension by oligonucleotide ligation. Typically, the oligonucleotides are fluorescently labeled and can be detected to determine the sequence of the template.

Some embodiments can utilize methods involving real-time monitoring of DNA polymerase activity. For example, nucleotide incorporations can be detected through fluorescence resonance energy transfer (FRET) interactions between a fluorophore-bearing polymerase and gamma-phosphate-labeled nucleotides, or with zero mode waveguides (ZMWs). Techniques and reagents for sequencing via FRET and/or ZMW detection are described, for example, in Levene et al. Science 299, 682-686 (2003); Lundquist et al. Opt. Lett. 33, 1026-1028 (2008); and Korlach et al. Proc. Natl. Acad. Sci. USA 105, 1176-1181 (2008), the disclosures of which are incorporated herein by reference.

### Paired-End Sequencing

As is known in the art, paired-end sequencing has many uses. For example, in certain circumstances, the amount of sequence data that can be reliably obtained from a target nucleic acid with the use of sequencing by binding techniques or sequencing by synthesis techniques, particularly when using blocked, labelled nucleotides, may be limited, e.g., to tens or a few hundred cycles of incorporation (and thus to tens or a few hundred bases in a sequencing read). While such short reads can be extremely useful, particularly in applications such as, for example, SNP analysis and genotyping, in many circumstances it is advantageous to be able to reliably obtain further sequence data for the same target molecule. The technique of “paired-end” or “pairwise” sequencing allows the determination of two reads of sequence from two places on a single polynucleotide duplex, e.g., from the two opposite ends of the duplex. In applications where the target duplex has a length that is more than twice that of the average sequencing read length, the knowledge that the “paired-end” sequences are known to occur on a single duplex, and are therefore linked or paired in the genome, greatly aids the assembly of whole genome sequences into a consensus sequence, for example. In applications in which the target duplex has a length that is less than twice that of the average sequencing read length, the reads from the opposite ends overlap in the middle of the target and can thus be compared to increase accuracy of base determination in this region, which is useful since accuracy can decrease as the sequencing techniques approach the limit of their read length. Additional uses of paired-end sequences are well known in the art.

An improved method of paired-end sequencing is provided herein. The following references provide alternative methods and uses of paired-end sequencing. See, for example, U.S. Pat. No. 8,192,930 “Method for sequencing a polynucleotide template” by Vermass, U.S. Pat. No. 8,105,784 “Paired end reads on libraries made by bridge amplification” by Rigatti, international patent application publication WO2004070005 “Double ended sequencing by blocking and unblocking” by Chen, and U.S. patent application publication no. 20210189483 “Controlled strand displacement for paired-end sequencing,” which are incorporated by reference herein in their entirety for all purposes.

### Compositions, Systems, and Kits

Compositions, systems, and kits related to, produced by, or of use in the methods are also features of the invention. For example, one general class of embodiments provides a composition that includes an array of nucleic acid concatemers. The concatemers are bound to a surface, e.g., with different concatemers at different sites in an orderly arrangement or with different concatemers at different sites in a disordered array. The location of a given concatemer within the array can be predetermined or random. Each concatemer comprises multiple copies of: a first adapter region comprising a first sequencing primer binding site, a forward strand of a target nucleic acid sequence, a second adapter region different from the first adapter region, and a reverse strand of the target nucleic acid sequence complementary to the forward strand. The second adapter region can include a second sequencing primer binding site that differs in sequence from the first sequencing primer binding site.

The concatemers can be covalently or noncovalently bound to the surface. For example, each concatemer can comprise a first member of a binding pair (e.g., biotin) that is bound to the second member of the binding pair (e.g., avidin or streptavidin) bound in turn to the surface. The array can include large numbers of concatemers, thousands to millions to billions (e.g., at least one million, at least ten million, at least 100 million, at least one billion, at least two billion, or at least three billion concatemers). The concatemers in the array can be immobilized, for example, on a planar surface of a substrate, on a non-planar surface of a substrate, or in a three dimensional manner within a substrate, such as within a gel matrix. Suitable substrates are well known in the art and include, but are not limited to a slide, a chip, a surface of a flow cell, and the like.

In some embodiments, a first sequencing primer is hybridized to the first sequencing primer binding sites. In some embodiments, a second sequencing primer is hybridized to the second sequencing primer binding sites. The composition can include nascent strands produced by extension of the first and/or second sequencing primer. Nascent strands produced by extension of the first sequencing primer are optionally blocked. The composition optionally also includes a polymerase (e.g., a strand-displacing polymerase or a polymerase that lacks strand displacement activity), one or more nucleotides (e.g., naturally occurring nucleotides, non-natural nucleotides, labeled nucleotides, reversible terminator nucleotides, and/or chain-termination nucleotides), a masking primer, a blocking oligonucleotide, a displacement primer, a masking strand, a displaced strand, one or more staple oligonucleotides, and/or other reagents employed in sequencing processes. The composition is optionally present in a nucleic acid sequencing system.

Essentially all of the features noted above apply to these embodiments as well, as relevant, e.g., with respect to number of copies of the repeating unit in the concatemer, inclusion of a nuclease in the composition for removal of nascent or masking strands or nicking of adapter regions, inclusion of single-stranded binding protein in the composition, suitable array substrates, and/or the like.

Another general class of embodiments provides a kit that includes in any combination one or more of the following: a solid support configured to bind a multiplicity of nucleic acid concatemers, a first stem-loop adapter, a second stem-loop adapter different from the first stem-loop adapter, reagents for performing rolling circle amplification (e.g., a rolling circle amplification primer, a strand-displacing polymerase, and one or more nucleotides), a first sequencing primer, optionally a second sequencing primer, and reagents for performing nucleic acid sequencing (e.g., a polymerase and one or more nucleotides, optionally including one, two, three, or four labeled nucleotides). Polymerases employed for rolling circle amplification and sequencing are typically different polymerases, but can in some embodiments be the same. The kit can also include additional reagents useful in producing a circular nucleic acid molecule, performing rolling circle amplification to produce a concatemer, and performing nucleic acid sequencing, including but not limited to buffered reaction solutions, a masking primer, a blocking oligonucleotide, a displacement primer, one or more staple oligonucleotides, a site-specific endonuclease, and/or an exonuclease. The kit typically also includes instructions for using the components for carrying out the desired processes, as also described or referenced herein, e.g., for producing a circular nucleic acid molecule, performing rolling circle amplification to produce a concatemer, and performing nucleic acid sequencing. Components of the kit are packaged in one or more containers.

Essentially all of the features noted above apply to these embodiments as well, as relevant, e.g., with respect to suitable array substrates, inclusion of single-stranded binding protein, and/or the like.

Various nucleic acid sequencing systems suitable for performing sequencing processes useful in the methods of the invention are known in the art (see, e.g., the references detailing sequencing techniques hereinabove) and/or are commercially available. Such sequencing systems can include a fluid handling system (e.g., for delivering reagents to a substrate comprising an array of concatemers during sequencing processes) and a detection system.

For example, the sequencing processes, e.g., using the substrates described above and the compositions and methods of the invention, can be exploited in the context of a fluorescence optical system that is capable of illuminating various positions on the substrate, and obtaining, detecting and separately recording fluorescent signals from these positions (e.g., individually resolvable positions each occupied by a different concatemeric template). Such systems typically employ one or more illumination sources that provide excitation light of appropriate wavelength(s) for the labels being used. An optical train directs the excitation light at the reaction region(s) and collects emitted fluorescent signals and directs them to an appropriate detector or detectors. Additional components of the optical train can provide for separation of spectrally different signals, e.g., from different fluorescent labels, and direction of these separated signals to different portions of a single detector or to different detectors. Other components may provide for spatial filtering of optical signals, focusing and directing the excitation and/or emission light to and from the substrate.

The methods described herein can further include computer implemented processes, and/or software incorporated onto a computer readable medium instructing such processes. As such, signal data generated by the reactions and optical systems described above is input or otherwise received into a computer or other data processor, and subjected to one or more of various process steps or components. Once these processes are carried out, the resulting output of the computer implemented processes may be produced in a tangible or observable format, e.g., printed in a user readable report or displayed upon a computer display. The resulting output may be stored in one or more databases for later evaluation, processing, reporting or the like, or it may be retained by the computer or transmitted to a different computer for use in configuring subsequent reactions or data processes.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes, including for the purpose of describing and disclosing devices, compositions, formulations, and methodologies which are described in the publication and which might be used in connection with the presently described invention.

1. A method for paired-end sequencing comprising:
a) providing a nucleic acid concatemer comprising multiple sequential copies of: a first adapter region, a forward strand of a target nucleic acid sequence, a second adapter region different from the first adapter region, and a reverse strand of the target nucleic acid sequence that is complementary to the forward strand; and
b) performing a sequencing process to produce paired-end reads of the target nucleic acid sequence by:
i) hybridizing a first sequencing primer to the first adapter regions, and obtaining a first read of a first portion of the target nucleic acid sequence by sequencing from the first sequencing primer; and
ii) hybridizing a second sequencing primer to the second adapter regions, and obtaining a second read of a second portion of the target nucleic acid sequence by sequencing from the second sequencing primer;
wherein the first read and the second read comprise paired-end reads of the target nucleic acid sequence.

2. The method of claim 1, wherein the nucleic acid concatemer is produced by:
providing a circular nucleic acid molecule comprising: a central region comprising the forward strand and the complementary reverse strand; the central region having two ends, the forward strand connected to the reverse strand at one end with a first connecting region, and the forward strand connected to the reverse strand at the other end with a second connecting region; and
performing rolling circle amplification using the circular nucleic acid molecule as a template to produce the nucleic acid concatemer.

3. The method of claim 2, wherein the rolling circle amplification step is performed in solution.

4. The method of claim 2, wherein the rolling circle amplification step is performed on the surface of a solid support.

5. The method of claim 1, wherein step ii) is performed after step i).

6. The method of claim 5, wherein, after obtaining the first read, nascent strands formed by sequencing from the first primer are removed before hybridizing the second sequencing primer to the second adapter regions.

7. The method of claim 6, wherein the nascent strands formed by sequencing from the first primer are removed by cleavage and washing, exonuclease digestion, or denaturation.

8. The method of claim 5, wherein the nascent strands formed by sequencing from the first primer are not removed.

9. The method of claim 8, wherein the 3′ ends of the nascent strands formed by sequencing from the first primer are blocked before hybridizing the second sequencing primer to the second adapter regions.

10. The method of claim 1, wherein steps i) and ii) are performed at the same time.

11. The method of claim 10, wherein sequencing from the first and second sequencing primers to obtain the first and second reads comprises detecting signals, wherein signals that contribute to obtaining the first read are distinguishable on the basis of their intensity from signals that contribute to obtaining the second read.

12. The method of claim 11, wherein the first and second sequencing primers are provided at different concentrations.

13. The method of claim 11, wherein the second sequencing primer is provided as a mixture of extendable and non-extendable oligonucleotides.

14. The method of claim 11, wherein the first sequencing primer and the second sequencing primer anneal to their respective adapter regions with significantly different efficiencies.

15. The method of claim 10, wherein sequencing from the first and second sequencing primers to obtain the first and second reads comprises mapping to a known reference sequence.

16. The method of claim 1, wherein sequencing from the first and second sequencing primers comprises extending the first and second sequencing primers with a strand-displacing polymerase.

17. The method of claim 1, wherein sequencing from the first and second sequencing primers is performed in the presence of a single-stranded binding protein.

18. The method of claim 1, wherein performing a sequencing process to produce paired-end reads of the target nucleic acid sequence comprises: synthesizing a first masking strand complementary to the forward strand prior to hybridizing the first sequencing primer to the first adapter regions, and synthesizing a second masking strand complementary to the reverse strand prior to hybridizing the second sequencing primer to the second adapter regions.

19. The method of claim 1, wherein sequencing from the first and second sequencing primers comprises performing a sequencing by binding technique.

20. The method of claim 1, wherein the nucleic acid concatemer comprises at least 100 sequential copies of the first adapter region, the forward strand, the second adapter region, and the reverse strand.

21. A method for nucleic acid sequencing comprising:
providing a nucleic acid concatemer comprising multiple sequential copies of: a first adapter region, a forward strand of a target nucleic acid sequence, a second adapter region different from the first adapter region, and a reverse strand of the target nucleic acid sequence that is complementary to the forward strand;
hybridizing a masking primer to the second adapter regions, and extending the masking primer to produce a first masking strand complementary to the forward strand, wherein the first masking strand is not also complementary to the entirety of the first adapter region; and
hybridizing a first sequencing primer to the first adapter regions, and sequencing from the first sequencing primer to obtain a first read of a first portion of the target nucleic acid sequence.

22-35. (canceled)

36. A composition comprising:
an array of nucleic acid concatemers, each bound to a surface and comprising multiple copies of: a first adapter region comprising a first sequencing primer binding site, a forward strand of a target nucleic acid sequence, a second adapter region different from the first adapter region, and a reverse strand of the target nucleic acid sequence complementary to the forward strand.

37-43. (canceled)
